(19) United States
(12) Patent Application Publication
(10) Pub. No.: US 2012/0231545 A1
(43) Pub. Date: Sep. 13, 2012

(54) METHOD FOR MODIFYING BOVINE EMBRYO STEM CELLS AND METHOD FOR PURIFYING PROTEINS PRODUCED BY MODIFIED BOVINE EMBRYO STEM CELLS

(76) Inventor: Sharon Lisauskas Ferraz de Campos, Lago Norte (BR)

(21) Appl. No.: 13/256,340

(22) PCT Filed: Mar. 5, 2010
(86) PCT No.: PCT/BR10/00062

§ 371 Date: Dec. 4, 2011

(30) Foreign Application Priority Data
Mar. 6, 2009 (BR) PI 0903975-9

(51) Int. Cl. C12N 9/64; C12N 15/85

(52) U.S. Cl. 435/455; 435/226

(57) ABSTRACT

The present invention relates to the modification process of bovine embryonic stem cells and purification process of proteins generated by modified stem cells. In particular, the present invention lies in the field of medicine and veterinary.

### FIELD OF THE INVENTION

The present invention relates to the modification process of bovine embryonic stem cells and purification process of proteins generated by modified stem cells. In particular, the present invention lies in the field of medicine and veterinary.

### PRIOR ART

The growing demand for bio-molecules of pharmacological interest has led to the development of additional systems for the production of recombinant proteins in large scale and at lower costs. Expression systems using higher prokaryotes and eukaryotes cells have been identified as possible alternatives for the production of proteins of pharmaceutical interest (Leite, 2000). The secretion of these polypeptides in the milk of transgenic animals also enables not only high yields of heterologous pharmaceuticals production as well as the occurrence of processing and post-translational modifications in the right way (Kerr et al., 1996). In a complementary way, the production of this type of biomolecule in eukaryotic systems have shown interesting features both with respect to the maintenance of rheir synthesis, folding and processing process, and the significant cost savings, increased stability, maintenance of biological activity and lack of contaminants and pathogens common to humans (Kusnadi et al., 1997). Blood clotting factor IX (FIX) is a practical example of this heterologous production, which has been proven to detect recombinant and biologically active factor IX when produced in the milk of transgenic mice (Lisauskas et al., 2008). Mus muscullus transgenic mice were developed as an experimental model in Brazil transformed with the human gene of the blood clotting factor IX (FIX). The mice secreted the recombinant FIX in milk. Activity tests of the recombinant protein in blood samples of type B hemophilic patients were performed with the aid of coagulometric, confirming the bioactivity of the protein. The expression of biologically active blood clotting human factor IX in transgenic mice (Eyestone, 1994; Lisauskas et al., 2008) justifies the applicability of the use of eukaryotic heterologous systems, used as bio-factories to the steady production, large-scale and under low cost levels of human proteins, limited by the scarcity of biological supply, i.e., the initial raw material for subsequent purification of these proteins.

The first successful uses of recombinant DNA technology on the production of proteins in heterologous systems have been demonstrated through the commercial production of hormones and vaccines in micro-organisms and/or cells in culture. Human proteins like insulin, antibodies, erythropoietin, factor VIII, factor IX, etc., can be produced in “artificial” and substitute system, in relation to the techniques of purification from human compounds. These systems can serve as a constant production chain of new drugs associated with recombinant human proteins. Although effective, this strategy has some inherent limitations. Among them, the main one is the impossibility of mass producing. This is mainly because the fermentors contain a limited number of cells, which are kept in sub-optimal metabolic conditions. Fermenters with high capacity can produce recombinant proteins in large scale, but not at low cost.

In an attempt to technological advances in recombinant production, have emerged additional technologies for the production of pharmaceutically valuable proteins wherein the heterologous systems were restricted to the culture of in vitro mammalian cells in vitro or production in microorganisms. From the 90's, the use of transgenic animals has been shown to be highly efficient for the production of recombinant proteins in milk, at large-scale and low cost (Houdebine, 1994; Wall, 1996).

Using a transgenic mouse expressing a recombinant protein has validated the hypothesis of using animals as bioreactors (Palmiter et al., 1982, Choo et al. 1987; Lisauskas et al., 2008). For large-scale production, the demonstration was given in 1987 through a sheep producing beta-lactoglobulin (Simons et al., 1987). These results were confirmed by experimental production of dozens of recombinant proteins in the milk of different animal species (Clark, 1998; Wall, 1999; Rudolph, 1999).

The possibility of using transfected somatic animal cells (Feigner et al. 1989; Egilmez et al., 1996, Friend et al., 1996, Oliveira et al. 2005; Iguma et al., 2005) as donors of nuclei for nuclear transfer has opened a new practical and viable approach for generating transgenic bovines (Carver et al., 1993). McCreath et al. (2000) produced a transgenic sheep from the nuclear transfer of fetal fibroblasts, transformed with the linearized COLT-2 vector, which induced the expression of the alpha-antitrypsin protein in the milk of sheep. However, many technical challenges still need to be overcome and processes must be developed and optimized in the field of nuclear transfer, introduction into the genome and expression of transgenes to desired level (Palmiter et al., 1982, Simons et al. 1987; Clark, 1998; Wall, 1999; Rudolph, 1999; Lisauskas et al., 2007).

In order to improve the process of generating transgenic bovines, Saito et al. (2003) and Wang Li et al. (2005) genetically engineered bovine embryonic stem cells and achieved superior results in the production and development of embryos after nuclear transfer, compared with the use of genetically modified somatic cells (fibroblasts) as donor of transgenic nuclei.

Public health is a major focus of biotechnology through the use of genetic engineering in the production of bioproducts, foods with nutraceutical features, biopesticides, DNA vaccines, and human recombinant proteins obtained from alternative production systems. Today, Brazilians' life expectancy tends to increase due to advances of biotechnology in medicine, through early diagnosis of lethal diseases; in agriculture, by increasing productivity in plantation areas and through genetic improvement of commercial cattle; and in industry, through the development of new drugs with applications in the medical field, so that the limiting factors such as supplies of plasma and human biological compounds are supplied by alternative systems of production of drugs.

A transgenic animal, as defined by the Federation of Laboratory Animal Science Associations (FELASA), is “an animal that has its genome artificially modified by man, caused by introducing, modification or inactivation of a gene (a sequence set of DNA). This process will culminate in alteration of genetic information contained in every cell of the animal, even in the germ cells (ova and spermatozoon) causing this modification to be passed to descendants.”

The most commonly used method to introduction of genes is by adding, through which is inserted into a genome or multiple copies of a gene of interest—hence its other names: adding gene and overexpression of genes model. The added gene may be endogenous or exogenous. The first type already exists in the animal's genome. This is used when one wishes to produce a greater amount of existing protein encoded by increasing the amount of the copy in the genome. Exogenous genes belong to another species and are used to make an animal to produce a new protein, absent in the species receiving the desired shape.

The first successful use of recombinant DNA technology in the production of proteins in heterologous systems has been demonstrated through the commercial production of hormones and vaccines in micro-organisms and/or cells in culture. However, there are limitations to the production of some recombinant proteins in bacterial systems is not due to post-translational processing in order to produce biologically active molecules. In other cases, the proteins of interest are in the form of aggregates, and can not be easily retrieved. Alternatively, different recombinant proteins have been produced in animal cells. Fermenters containing hybridoma, CHO cells among others, have been extensively used for the production of antibodies against hepatitis B, erythropoietin, factor VIII etc. Although effective, this strategy has some inherent limitations. Among these, the principal is unable to mass production. This is mainly because the fermentors contain a limited number of cells, which are kept in sub-optimal metabolic conditions. Fermenters with high capacity can produce recombinant proteins in large scale, but not at low cost.

In an attempt to technological advances in production of recombinant proteins, there are additional technologies for the production of proteins of economic interest in large-scale production. Transgenic animals (Wall, 1996; Houdebine, 1994) and transgenic plants (Kusnadi et al. 1997, Leite et al., 2000.) have been shown to be highly efficient for the production of biologically active recombinant proteins in large scale and under lower costs.

Using a transgenic mouse expressing a recombinant protein has validated the hypothesis of using animals as bioreactors (Lisauskas et al., 2008, Choo et al., 1987, Simons et al., 1987, Palmiter et al., 1982). For large-scale production, the demonstration was given in 1993, through the production of recombinant proteins in ovines (Carver et al. 1993; Wright and Colman, 1997). These results were confirmed by experimental production of dozens of recombinant proteins in the milk of different animal species (Rudolph, 1999; Clark, 1998; Wall, 1999; Eyestone, 1994).

Milk contains proteins that are naturally expressed by the mammary gland during lactation. The two main categories of proteins produced in milk involve the caseins and soluble proteins. The coding genes for some of these proteins were cloned and the regulatory elements that control the sites of tissue-specific and time expression were characterized. In our constructed expression vector, the coding sequence of the gene of interest, the gene of blood clotting factor IX was built connected to a promoter that directed the expression of the gene product to the mammary glands of mice.

In this project, the Laboratory of Embrapa Genetic Resources and Biotechnology (CENARGEN)—Brasilia—DF, in partnership with the University of Brasilia—UnB and Federal University of Sao Paolo-UNIFESP, developed transgenic mice expressing human clotting factor IX in the milk. The genetically modified mice were produced based on the technique of microinjection of the expression vector directly into the male pronuclei, producing genetically modified germlines (Lisauskas et al., 2008). Studies were conducted to evaluate the presence of the transgene inserted both in the matrix and in the progeny using PCR and southern blot, and analysis of factor expression in the milk of mice (western blot). The validation of this heterologous system through the production of this recombinant protein was confirmed by testing the bioactivity of the recombinant protein purified from milk, demonstrating its clotting ability and activity of the protein (human factor IX) in the blood of hemophiliacs. Hospital de Apoio de Brasilia was responsible for the bioassays of the recombinant protein.

After the validation of the heterologous system of production of recombinant proteins produced in the milk of animals generated as experimental models, the next step was to achieve large scale production. Transfected and properly characterized animals cells have been used as donor sources of their nuclei for the synthesis of proteins in large animals. In this context, the species of caprines and ovines are major candidates for production of recombinant protein due to high capacity of milk and consequently recovered protein production (McCreath et al. 2000; Yull et al., 1997).

The possibility of using somatic cells transfected animals—cattle fibroblasts—(Iguma et al. 2005; Egilmez et al., 1996, Friend et al. 1996; Feigner et al., 1989) as donor nuclei for nuclear transfer opened new perspectives for the generation of transgenic sheep and cattle (Cibelli et al., 1998, Carver et al., 1993). McCreath et al. (2000) produced a transgenic sheep from the nuclear transfer fetal fibroblasts, transformed with the vector linearized COLT-2, which induced the expression of the protein alpha-antitrypsin in the milk of sheep.

However, many technical challenges still need to be overcome and processes must be developed and optimized in the areas of: election cells as donors of transgenic nuclei for nuclear transfer; introduction into the genome and expression of transgenes to desired level, increased rates of rebuilted embryost and pregnancy rates of transgenic embryos (Lisauskas et al. 2007; Iguma et al. 2005; Rudolph, 1999; Wall, 1999; Clark, 1998). The acquaintance of the phenomena involved in the integration and segregation of exogenous genes in transgenic animals, particularly in the large ones, is crucial for the future control of these processes (Stinnakre et al., 1999).

In order to improve the process of generating transgenic animals, Saito et al. (2003) genetically modified bovine embryonic stem cells and achieved superior results in the production and development of embryos after nuclear transfer when compared with the use of somatic cells (fibroblasts) genetically modified as donors of transgenic nuclei.

This paper proposed the genetic modification of bovine cells to be studies models for future generation of lineages of donor cell suitable for producing transgenic cattle. Based on the strategy of transfection of fibroblasts isolated from adult tissue (Oliveira et al. 2005; Oleskovicz et al., 2004) MDBK (Madin Darby Bovine Kidney) bovines cell lineages were generated with non-deleterious insertions of transgene and variable expression levels (Fujiwara et al., 1999, Fujiwara et al. 2003; Forsbach et al. 2003; Lisauskas et al., 2007). The sites of integration of transgenes in the chromosomes that promote its expression are not well characterized but is believed to be transcriptionally active regions of euchromatin (Day et al., 2000). In the state of heterochromatin transcription factors are inaccessible, and often is related to hypermethylation of cytosines and hipoacetilaçäo by histones (Ng and Bird, 1999). The direct consequence of this chromatin configuration is that transgenes that integrate randomly in the vicinity of heterochromatin often have lower levels of expression (Dobie et al., 1996). With the studies done in this work, we could establish clones transfected lineages of bovine MDBK cells generating elite events in relation to integration sites of the transgene in the genome, and in relation to the detection of stable expression of these transgenes in these lineages. The future application of this characterization can serve as a model for integration of other expression vectors in the bovine genome, using target genes (“gene targeting”), for genetic transformation of cells to genetic transformation of transgenic nuclei donor cells for embyonic reconstruction after nuclear transfer. According to Iguma et al. (2005) nuclear transfer is based on the generation of transgenic animals with predetermined genotype. Bovine clone animals are produced from genetically modified cells, used as donor sources of transformed nuclei and microinjected in oocytes previously enucleated. The generated transgenic embryo goes to transfer in receptor cows (Cibelli et al. 1998; Schnieke et al. 1997, Campbell et al., 1996).

However, in order to increase the efficiency of producing transgenic embryos reconstructed after nuclear transfer (NT) and increase pregnancy rates of these embryos, the strategy that should be used is the transfection of stem cells isolated from bovine embryos (Li Wang et al, 2005). According to Saito et al. (2003) seven embryos from stem cells genetically modified were transferred to seven receptors animals and five fetuses (71%) were confirmed. The frequency rate of pregnancy (71%) and of animals born from blastocysts derived from genetically modified stem cells were higher than the rates of pregnancies generated from somatic cells (approximately 55%) reported after the NT. The development of systems for the generation of transgenic bovines (Saito et al. 2003; Li Wang et al., 2005) aim to improve the production of transgenic embryos using bovine embryonic stem cells as genetically modified nuclei donors. In our study, five colonies of bovine embryonic stem cells were established to be transfected in vitro in the future, and act as transgenic nuclei donor sources, in order to increase both the rate of post-NT embryonic rebuild and post-pregnancy rates embryo transfer (ET).

The use of transgenic animals should allow the expression of proteins with complex structures, stability and biological activity in vitro and in vivo production, consistency of production between generations and between different animals, and enables the production of proteins that have not been produced using other systems. One can achieve an efficient production of tens of grams of recombinant protein per liter of milk, reaching kg per year. An increase in productivity between 10-1000 times, large-scale production in a more economically way, and low investment compared to cell culture systems. Production systems that use cells in culture can produce a maximum of 2.1 grams, or more typically 100-200 mg per liter per day (Wright and Colmann, 1997, Van Cott et al., 1996, Denman et al. 1991; Gordon et al., 1987). Compared with a goat: its daily production of milk is typically 2-4 liters containing 10 grams or more of recombinant protein per liter, which may provide more raw material than a recombinant protein of 1000 liters bioreactor (Denman et al., 1991).

Currently, the use of transgenic animals has been shown to be highly efficient for the production of recombinant proteins in milk, at large-scale and lower costs, which have been accepted by regulatory agencies responsible for the production of drugs. The main technology platforms exist regarding the use of animals as bio-factories are restricted to some research institutes and private companies located in the United States and Europe. In Argentina, five genetically modified bovine animals with the gene for human growth hormone produced in the milk were generated by the private company Bio Sidus in the period 2002 to 2004. According to the company, a single animal can produce 15 grams of protein per day and half a kg per month. Each dose received by a patient contains little more than 1 mg of growth hormone of maximum purity. The establishment of the systems required for effective production of recombinant proteins in the milk of transgenic animals in Latin America as one of the technological strategies.

The use of transgenic technology for production of recombinant proteins should provide a reliable, renewable and effective source for the production of biologically active proteins, which would only be available through the use of blood cells, tissues or bioreactors. The use of transgenic technology is especially attractive for the production of recombinant proteins required in large scale. This is due to units of required dosages, multiple administrations, large-scale application in the population, need for industrial production. In addition, the use of transgenic animals allows to express biochemically complex proteins that can not be produced in an economically viable way in cell culture. The significant low-cost in capital and operating costs for the production of raw compounds, cause the transgenics having a more cost effective than other systems for recombinant protein production.

Some of the advantages of producing recombinant proteins using transgenic are: milk as a source of na easy to obtain, good public acceptance, renewable, abundant, safe and unprocessed raw material; the possibility of production of heterologous proteins with complex structures; protein stability and activity biological in vitro and in vivo; consistent production between the various generations of animals; production efficiency: tens of grams per liter of raw milk, many kilograms of protein per animal (goat, sheep, cattle) for years, productivity levels 10 to 1000 times higher than in cultured cell systems, recovery and purification of the protein in large quantities and high concentration of protein in the initial raw material/kg of milk.

In this context, the bovine species is chosen for the production of recombinant proteins due to the high level of milk and protein production, both accepted as dietary sources. The milk of bovines has been extensively characterized in a biochemical level, a fact that facilitates the development of subsequent steps for purification of recombinant proteins. In addition, there is the possibility of developing transgenic species for specific applications, such as cows for production in large quantities (tons) or rabbits and mice for the production of small amounts of required proteins on a smaller scale, or simply as a study model for expression of recombinant proteins in heterologous systems (Lisauskas et al., 2007).

The mammary gland can be considered the best currently available bioreactor (Colman, 1996; Rudolph, 1999). Extensive studies have demonstrated the possibility of producing a wide variety of recombinant proteins in milk, many of them being complex proteins, such as human IGFI (Zinoveiva et al., 1998), hGH (Devinoy et al., 1994), human lysozyme (Lee et al., 1998), human lactoferrin (Platenburg et al., 1994), human erythropoietin (Sohn et al., 1999), human parathyroid hormone (Rokkon et al., 1996). These examples demonstrate the ability of the mammary gland to synthesize and secrete mature recombinant proteins.

In economic terms, there is an expanding global market for different classes of recombinant proteins expressed in milk. Sometimes demand exceeds 100 kg. The annual market for antithrombin III, used to treat deficiency (hereditary), is approximately 70 kg at a cost of $2000-6000 per gram, according to IMS America Ltd. (Plymouth Meeting, Mass.). In the case of alpha-antripsina, indicated for the treatment of cystic fibrosis, the market is close to U.S. $30-50 million, of which 350 kg are used in the U.S. alone, according to IMS. For proteins from human plasma, factor VIII and IX, the annual market is about 5-10 kg. According to World Federation of Hemophilia (Montreal, Canada), the current sale of factor IX is approximately U.S. $190 million in the United States; factor VIII is approximately U.S. $2 billion. The potential market for the products mentioned has been limited by the supply of plasma. In Brazil, according to the Support Hospital of Brasilia—DF, any factor that is purchased in the country is imported. A bottle of medicine costs about $100, and each patient gets to use 20 bottles per week.

The final step, which consists in the purification of recombinant proteins in milk usually does not present particular difficulties. Possible biological contaminants are the prions that may present problems. However, there is evidence approved, including the World Health Organization, emphasizing that prions have been detected in association with milk and semen (Rudolph, 1995; Ziomek 1996). Moreover, recently, livestock producers of recombinant proteins targeted marketing require closed systems of sanitary control in order to eliminate possible contamination by prions from these controlled animals (Gavin, 2001).

This technology platform will form the fundamental basis for the future use of different regulatory and coding, prospected and properly characterized sequences, through projects of functional genomics analysis and proteomes. As a direct consequence, a scenario will enable effective application of the manipulation of recombinant DNA technology to generate products for the benefit of our society. Hemophilia in Brazil and in the world

Hemophilia is a bleeding disorder resulting from the inability of the blood clot. It reaches the male of any ethnic group in the proportion of 1 per 10 000 births. In Brazil, about 8,000 hemophiliacs registered, however, due to the lack of diagnosis, patients are not yet identified.

The blood of the hemophiliac produces inadequate amounts of clotting factors such as Factor VII, VIII and IX, among others. Hence the need to take venous applications to stem the bleeding, which has effective action in the body for about 12-24 h, according to each case. The traditional replacement form is the factors of plasmatic origin, in other words, the products derived from blood donations. However, recent research led to the discovery of non-blood products, also known as recombinant, and with the same therapeutic effects.

The availability of concentrates to recombinant clotting factors has allowed the realization of effective treatments for the prevention of sequelae of haemophilia without increasing the risk of viral contamination once existed with the use of products derived from human blood. The inclusion of the drug-free human plasma is the only way currently available to eliminate the risk of exposure to not studied or unknown new pathogens.

It is worth noting the contamination of catastrophic proportions in the world of the HIV infection, which struck susceptible groups such as community of hemophiliacs in the 80's. It is also worth remembering the existence of more recent infectious agents as the “Creutzfeldt-Jakob”, related to bovine spongiform encephalopathy, known to all as mad cow disease. Featured also is the virus “H5N1 avian influenza”, which causes bird flu.

The risk of contamination by pathogens transmitted by human plasma has been reduced by investment in technologies for viral inactivation in order to make the drug less risky applications. However, this process burdens the cost of production and induces the industrial park of drugs to invest in recombinant products.

The continuing investment of resources in research promotes competitive advantage held by the holder of knowledge. There are countless examples of those who invest high amount of resources on a particular line of research, to then make a profit on the sale of their products in use licenses or other forms of investment return. In the specific case, the field of research for the production of recombinant factors tends to become plausible and worthwhile investment, since the following are taken into account:

1) Brazilian law does not allow effective monitoring of the donor, limiting the quantity and quality of raw material (human blood), a fact that reduces the service demand, it is worth noting that the supply is limited also in other countries;

2) the product not derived from human blood significantly reduces the possibility of allergic reactions, frequent in many hemophiliacs patients, making treatment more difficult and increasing the safety of infusion;

3) to improve treatment of people with hemophilia is directly proportional to the amount of clotting factor supply, which can be enhanced by the inclusion of recombinants;

That said, there is urgent need to seek alternative sources of production of recombinant to contribute for treatment of coagulopathy, joining a select group of domain knowledge of fine chemicals, to enhance the Brazilian researcher and attain self-sufficiency.

There is an unpublished research in Brazil, suspended for now, which is the production of recombinant coagulation factor IX from the cow's milk. Proved to be a viable project, except counterproofs not yet submitted, in front of other similar research. It is unprecedented in the world. Its industrial-scale production ensures self-sufficiency in the domestic market with export possibilities. Since it is high technology medicine, the recombinant product can be traded freely on the market. In addition to Factor IX, can be produced by the same technology others like VII and VIII. The climate, environmental conditions and technology achieved by the Brazilian agricultural guarantee unlimited supply of raw material to be industrialized.

Hemophilia is a genetic coagulation disorder that prevents the blood to form an effective clot. In the absence of clot, exogenous internal injuries and external lacerations are unable to be resolved correctly. Hemophilia A and B are genetically inherited as recessive disorders linked to sex (X chromosome) and occurs almost exclusively in men in the order of 1 in every 5000 born in the United States and Canada. Approximately 13,500 Americans have hemophilia A (also called classic hemophilia), in which the clotting factor VIII is missing or is not produced in sufficient quantities. Already 3,500 people in the United States have hemophilia B (also called Christmas disease), in which the clotting factor IX is absent or not present in sufficient quantities. In Canada, about 2000 people have hemophilia A and 450 have hemophilia B. In Brazil, there is an estimated incidence of 1 in every 10,000 inhabitants, 85% of patients having hemophilia A and 15% having hemophilia B.

While hemophilia can result from a spontaneous genetic mutation, is most often presented as a hereditary disorder. This disease is caused by a deletion or mutation of a gene that modifies the body's ability to produce sufficient quantities of factors to promote clotting. This cascade acts in two ways, and aims the formation of a clot when a vessel is damaged, preventing the blood to indefinitely spill. People affected by this disease do not produce one of two important factors necessaries to this cascade be effective, the factor VIII or factor IX. Thus, they are unable to clot blood.

Until now, the treatment for this disease is the injection of these missing factors (the VIII or IX), through blood derived from healthy donors or produced by genetic engineering. These factors, however, are very unstable, requiring frequent injections.

Hemophilia is characterized as severe when the activity of the involved coagulation factor is less than 1% of normal. This often results in spontaneous bleeding. The disease is mild when the activity is greater than 5% of normal and moderate when the activity of coagulation factor lies between these two values. About 50% of hemophilia patients have moderate or severe disease and require treatment for severe bleeding from several times a month until a few times a year.

Typically, patients suffering from hemophilia B are treated with factor IX obtained from human plasma of healthy patients. But the administration of blood derivates in patients presents high risk of contamination by infectious agents such as hepatitis, AIDS, bacterial infections and parasites. Due to this reason, in many countries is increasing significantly the use of products produced by genetic engineering. In Canada, for example, a law requires, since 1995, only blood clotting factors of this type are marketed at the expense of blood derivates.

Until the 90s, the technologies for the production of recombinant proteins of pharmaceutical interest were restricted to the culture of mammalian cells in vitro or production in microorganisms. From there, the use of transgenic animals has proved highly efficient for the production of recombinant proteins in milk, large-scale and lower costs (Houdebine, 1994; Wall, 1996).

There are several applications of transgenic animal technology. According to Axelrod et al. (1990), it was shown that primary cells of fibroblasts from the skin of hemophilic dogs transfected by retrovirus containing cDNA of coagulation factor IX, secreted high levels of biologically active factor IX in the growth medium in which cells were being grown. Choo et al. (1987) produced transgenic mice by microinjection of human factor IX cDNA in pronuclei of pre-fertilized oocytes. The transgenic mice expressed high levels of mRNA and activity of active clotting factor, which were indistinguishable from the activity of factor IX from normal human plasma.

These data demonstrate the ability to express highly complex recombinant proteins in transgenic mice. Besides serving as background for the production of human factor IX in large animals (Schnieke et al. 1997, Cibelli et al., 1998) and large-scale production, and for therapeutic application of gene therapy for hemophilia B.

### Insertion of Exogenous DNA

Exogenous DNA can be inserted into the genome by retroviral integration, transposons, and for non-viral gene transfer techniques, such as transfection assays. The locations of these insertions are usually identified by preparation of genomic libraries and isolation of clone lineages containing the exogenous DNA clones and the flanking sequences. In this project, bovine transgenic cell lines were used as a model for studies of integration of transgenes. Several transfected MDBK (Madin Darby Bovine Kidney) clones cell lineages were expanded from a single isolated cell and placed in cell culture until the expansion to approximately 36 μg of DNA. The pre-established cell lines were plated at a concentration of 3×10⁵ cells/mL in a plate of 24 wells and transfected with the plasmid vector pCIneo-β, previously constructed in the laboratory, including the neo gene (neomycin antibiotic) and β-gal (β-galactosidase) under control of the constitutive promoter CMV (cytomegalovirus). Transfection was performed using Lipofectamina™ reagent (Invitrogen, USA), according to Oliveira et al., 2005. Twenty days after transfection and selection in supplemented medium with geneticina antibiotics (G418: 400 μg/mL), the transgenic cells were isolated and expanded in culture. Genomic DNA of clone lineages were rescued and the flanking sequences were identified by the technique of plasmid rescue in Escherichia coli.

Since the integration of exogenous DNA by transfection technique occurs randomly, the method based on the technique of IPCR (inverted PCR) to amplify the region flanking chromosomal DNA at the insertion site of the transgene is a simple and useful method for this purpose. The use of this technique by means of important studies should contribute to new knowledge in the construction of bovine vectors target genes.

Target genes are a powerful and efficient method of genetic manipulation and require essentially the same procedures for transfection, selection and cell culture. The object of reach for many biomedical benefits, such as antigen ablation of xenorreativos transplantation, inactivation of genes responsible for neuropatogenic diseases and the insertion of transgenes needed to produce certain proteins in human therapy, have prompted both commercial and medical sectors to investment in this new technology, whose proposal is based on the appropriate integration site in a more accurate way and therefore more efficient to produce certain variants in quantities desired.

### Expression Vectors

The key t of transgenesis technologyis summarized in the introduction of specific genetic information with new functionality into the host genome. The strategy for the construction of a “transgene” involves selecting a gene regulatory element (usually called a promoter, but usually containing an enhancer element and a promoter) that aims to determine the tissue in which the gene will be expressed, as well as the time and magnitude of expression. In some cases, the regulatory element can act as a “switch”, allowing the transgene to be “on” (activated) or “off” (inactive). The second part of the building is the gene DNA sequence that encodes the desired protein (typically referred to as the structural component of a transgene). The completion of the transgene is restricted to the regulatory element called “terminator” which has the function to “advise” the termination of transcription of the transgene in question. Most terminators consist of a rich sequence of A (Adenine nucleotide), called poly (A) tail, responsible for “signaling” to mechanism of transcription of that sequence is the end of the transgene.

### Cultivation of Animal Cells

Tissue culture has emerged in the early twentieth century (Harrison, 1907; Carrel, 1912) as a method to study the behavior of animal cells devoid of systemic changes occurring in the animal organism. These cells vary in number and size both during homeostasis, when under stress conditions. The technique was developed primarily by the breakdown of tissue fragments, since the cell growth was limited to its migration from the dissociated tissue fragment. The culture of a tissue may have its origins in the coupling of biological material, treated in different ways (enzymatic, mechanical or chemical), and scattered cells of treated tissue can then be cultured in vitro for subsequent studies.

The tissue culture technology has been industriously adopted in many routines in medicine and industry. Chromosomal analysis of cells taken from the uterus through the technique of amniocentesis, not only can reveal genetic disorders of the fetus that is not even born and viral infections can be analyzed qualitatively and quantitatively by means of monolayer culture of host cells, toxic effects of certain pharmaceutical compounds as well as analysis of potential environmental pollutants that can be detected and measured by colony formation assays (Freshney, 1992).

When starting a tissue culture, some parameters must be adjusted, such as the physical-chemical control of environment of the cells: pH, temperature, osmotic pressure, O₂, CO₂, etc. maintaining a relatively constant and controlled physiological condition. Most growth medium still requires supplementation with serum, whose concentration varies depending on the tissue in question (Honne et al., 1975). It may also contain undefined elements, such as certain regulatory hormones and other substances. Gradually, the functions of the serum as a result are studied and are replacing it by more and more constituents (Olmsted, 1967).

The cell type chosen for cultivation should be based on the utilization of culture, i.e., the cells are designed to: genetic transformation, permanent cell lineages, donor nucleus for nuclear transfer, monolayer for co-culture etc. In addition, the laboratory must have a minimum of necessary equipment and aseptic conditions, before starting a tissue culture.

In the present study were cultured bovine in vitro somatic cell lineages. MDBK cells are considered to be immortalized, i.e., the cells are senescent, even when cultured in vitro, and may be subjected to more than 300 raises (Lisauskas et al., 2007). These cells are adherent, with easy maintenance and rapid growth, consistent with long periods of culture and interventions in the course of the required monitoring analysis.

### Genetic Modification of Mammalian Cells

Several technologies for the introduction and expression of gene constructions in different animals have been developed and refined. Accurate transport systems with small amounts of DNA are limited to (1) microinjection of DNA into the male pronucleus of fertilized oocytes, and used to produce transgenic mice (Lisauskas et al., 2008), rabbits (Rosochacki et al. 2002), ovines (Simons et al., 1988, Hammer et al., 1985), suines (Hammer et al., 1985) and bovines (Wall et al., 1985, Wall et al. 1988; Krimpenfort et al. 1991, Wall et al., 1996), even with only a small proportion (−5%) of animals containing the transgene integrated into its genome (Eyestone, 1994; Damak et al., 1996); (2) the use of spermatic cells as exogenous DNA carrier sources (Gandolfi et al., 2000, Perry et al. 1999; Lauria & Gandolfi, 1993; Lavitrano et al., 1989, Brackett et al., 1971); and (3) vector-based in transposons (Belur et al., 2003). On the other hand, systems that carry large amounts of DNA involve the use of retroviral vectors (Chan et al. 1998; Haskell & Bowen, 1995, Huszar et al. 1985; Jahn et al., 1985; van der Putten et al., 1985) due to its efficiency in cell penetration and stable transgene integration into the host genome. Since retroviruses can only infect cells in cell division, may cause deleterious effects in cells as a result of insertional mutagenesis when the integration of the provirus in genome (Onions et al., 1993), more efficient systems of transport of DNA called adenoviral vectors that can infect cells that are not in cell division can also be used, although they cannot integrate themselves into the host genome (Channon et al. 1996; Tsukui et al. 1996; Feldman & Isner, 1995). Other viral vector systems, called episomes vectors, using the viral genome of a bovine papillomavirus typel and remains as stable extracromossomal genetic element of self-replication (Dimaio et al. 1982; Ohe et al., 1995, Sarver et al., 1981; Mannik et al., 2003).

Currently the safest and simplest required method to transfect mammalian cells is lipotransfection using cationic liposomes (Cibelli, 1998; Keefer, 2001, Oliveira et al., 2005). The mechanism of transfection mediated by liposome appears as a result of the association between DNA and cationic lipids that provide a net positive charge around the vector, allowing the connection to the net of negatively charged cell surface (Feigner et al., 1989). Liposomes are translocated into the cell by fusion with the plasma membrane or by phagocytosis (Wrobel & Collins, 1995, Friend et al., 1996), and some particles may enter the nucleus by fusion with the nuclear envelope (Wrobel & Collins, 1995; Friend et al., 1996, Thierry et al., 1997). Thus, bovine clone animals can be produced from characterized lineages of genetically modified cells as donor sources of transformed microinjected in previously enucleated oocytes nuclei (Campbell et al. 1996; Schnieke et al., 1997; Iguma et al., 2005).

### Locus of Integration

Due to the randomness of the integration of the transgene in the bovine genome by technique of lipotransfection, only a small number of events will generate transgenic animals, expressing the protein of interest at desired levels (Schnieke et al., 1997). To improve the method of lipotransfection, some authors have studied the locus of integration of plasmid vector into the host genome (Wu Shi-Li et al., 1996, Lisauskas et al., 2007), as well as the characterization of these sites. The approach is based on the technique of plasmid rescue in Escherichia coli and permits the withdrawal of the expression vector of the genome under study, and the characterization of flanking regions of host DNA in order to isolate the regions of integration of transgenes (Mannik et al., 2003).

The advantage of studying the locus of integration of the transgene allows for the selection and generation of elite events that express the transgene at higher and desirable levels besides the use of suitable lineages for nuclear transfer to generate transgenic bovines. The success reported by McCreath et al. (2000) is based on strategies developed for the application of the technique in the generation of bovines vectors with target genes (“gene targeting”).

In this work, pre-established parameters of lipotransfection for bovine somatic cells (Oliveira et al. 2005; Oleskovicz et al., 2004) were used as the base protocol and applied in MDBK bovine cell lines (Lisauskas et al., 2007) in order to to study the local integration of transgenes to serve as a model for genetic transformation of cells capable of being donor transgenic nuclei for nuclear transfer.

### Stem Cells

Stem cells have that name by having the ability to regenerate blood cells and various types of tissues that form the human body. For example, skin cells can only be but the skin, stem cells can form different tissues.

Every multicellular organism is composed by different cell types. Among the approximately 75 trillion cells exist in a grown man, for example, are found around 200 different cell types. They all derive from precursor cells, called stem cells (“stem cells”). The differentiation process that generates the specialized cells—skin, bone and cartilage, blood, muscles, nervous system and other organs and tissues—is governed in each case, the expression of specific genes in cells stem, but it is not known in detail how this occurs and which other factors are involved.

Stem cells are undifferentiated cells that can multiply and regenerate damaged tissues because they have the ability to transform into identical cells of the tissues in which they were deployed. For example, if a person with myocardial infarction has a part of the heart affected and cells in this region die, the stem cells can turn into heart cells and replace dead cells, regenerating damaged tissue. Another hypothesis, in the case of diabetes, is that stem cells, when inserted in the pancreas could differentiate and begin producing insulin, which would bring healing to people with this disease.

Stem cells are present from embryonic life to adulthood, and probably even our death. They are responsible for the formation of the embryo and also the maintenance of tissues in adult life. They are present in: various human tissues (blood, bone and other tissues), but the amount is very small; in umbilical cord and placenta (in larger quantities); in embryos in the early stages of cell division, i.e., in blastocyst phase.

### Morphological Classification

Totipotent: embryonic stem cells that can form all tissues including the placenta are called embryonic-totipotent. They are the first group of up to 32 cells, and form the first 72 hours after fertilization of the egg. Right now, this is not possible to identify in this cell group any specific tissue differentiation. The formation of the placenta and its attachments only occurs when these totipotent cells are implanted in the uterus.

Multipotent: Embryonic cells from the fifth or sixth day after fertilization, where they constitute a group of 64 cells, are capable of forming any kind of tissue except placenta, and are called multipotent cells.

Oligopotentes: adult stem cells that give rise to more than one type of tissue are called oligopotentes, and are, for example, in intestinal tissue.

Unipotentes: On organs already formed, for example, the nervous system, adult type stem cells are found that lead to a single type of tissue, the likely function of these cells is the repair of certain tissues. Already in the bone marrow adult type stem cells function is to keep the level of elements figured on blood cells that need constant replacement.

### Therapeutic Cloning (Yanagimachi R, 2002)

This process is to obtain an embryo of the sick person through the cloning technique and remove its stem cells. These cells have the potential to become any type of adult cell of the body, for example, heart or nerve cells. Thus, they could be encouraged to become the same type of cell that is injured in the body of the sick patient. For example, a person with leukemia who needed a bone marrow transplant would be cloned, giving rise to an embryo, which would be removed from the stem cells. Thus, the person giving it to herself, without running the risk that the body would reject the transplant, because the cells used were taken from his clone, which would have the same genetic constitution.

The use of stem cells for therapeutic purposes may represent perhaps the only hope for the treatment of many diseases or for patients who suffer disabling spinal cord injuries that impede their movements. In 1997 it was announced the first mammal created from somatic cells of an adult through nuclear transfer, the sheep Dolly (Wilmut et al., 1997). Dolly was born only after 276 failed attempts. Moreover, among the 277 cells, of “the mother of Dolly” that were inserted into an enucleated egg, 90% didn't reach even the blastocyst stage. A later attempt to clone other mammals such as mice, pigs, calves, a horse and a deer also has shown a very low efficiency and a very large proportion of miscarriages and malformed embryos. It is interesting that among all the mammals that had been cloned, the efficiency is slightly higher in calves (about 10% to 15%). Penta, the Brazilian first calf cloned from an adult somatic cell in 2002 died just over a month. Also in 2002, they announced the cloning of the copy cat, the first pet cat cloned from an adult somatic cell. For this 188 eggs were used that produced 87 embryos and only one live animal. In fact, recent experiments with different animal models have shown that reprogramming genes to the embryonic stage, the process that led to Dolly, it is extremely difficult.

The group led by Ian Wilmut, the Scottish scientist who became famous by this experience, says that virtually all animals that have been cloned in recent years from non-embryonic cells are faulty. Among the various defects observed in the few animals that were born alive after numerous attempts, there is: shortened telomeres; abnormal placentas; gigantism in sheep and cattle; heart defects in pigs; lung problems in cows; sheep and pigs, immune problems; failure production of white blood cells; muscle defects in sheep.

According to Hochedlinger and Jaenisch (2003), recent advances in reproductive cloning allow four major conclusions: 1) Most clones die early in gestation; 2) cloned animals have similar defects and abnormalities, regardless of the donor cell or species; 3) these abnormalities probably occur due to failures in reprogramming of the genome; 4) the efficiency of cloning depends on the stage of differentiation of the donor cell. In fact, reproductive cloning from embryonic cells has shown a higher efficiency of 10 to 20 times probably because the genes that are critical in early embryogenesis are still active in the genome of the donor cell.

One of the objectives of animal biotechnology is the application of current techniques of genetic engineering for the production of large animals with desirable characteristics. Sheep (Dattena et al., 2006), pig (Piedrahita et al., 1990), rabbit (Chiang et al., 2008), cattle (Keefer et al., 1994, Verma et al., 2007) the rat (Ouhibi et al., 1995) and monkey (Mitalipov et al., 2003) have been reported as potential sources of donor stem cells.

The method used for the isolation of human embryonic stem cells has been adapted to other species with some modifications. However, preliminary attempts to culture the inner cell mass (ICM) of various species upon layer of mouse embryonic cells or on primary cultured of mouse fibroblasts in the presence of leukemia inhibitory factor (LIF) were rarely successful. A combination of growth factors may be required for the proliferation of these pluripotent cells, as demonstrated in cell culture of primary mice lineages (Tielens et al., 2006). In our study, bovine embryonic stem cells isolated from the inner cell mass of bovine blastocysts produced in vitro was only possible when grown on layer of embryonic stem cells of mice, providing in this way growth medium conditioned by such cells in concomitant growth.

In Brazil, with the prospect of low rates of embryo reconstruction and low pregnancy rates after cloning by nuclear transfer through the use of somatic cells led to the need to isolate stem cells from bovine embryos produced in vitro, as expected in the use of transfected nuclei for the future generation of transgenic bovines used as bioreactors.

The isolation technique of bovine embryonic stem cells opens a perspective for the future generation of viable clone lineages of stem cells for studies of gene function, gene expression and the generation of transformed nuclei with exogenous genes, mainly of pharmaceutical interest, such as donor sources of these nuclei for nuclear transfer and the generation of bioreactors animals.

It is extremely important that people understand the difference between human cloning, therapeutic cloning and stem cell therapy with embryonic or not stem cells. Most countries of the European Community, Canada, Australia, Japan, China, Korea and Israel have approved embryonic stem cell research from embryos up to 14 days. This is also the position of science academies from 63 countries, including Brazil. It is vital that our legislation also approve these researches because they could save countless lives.

These 63 science academies of the world that stood against the reproductive cloning advocate the embryonic stem cell research for therapeutic purposes. As for those who believe that therapeutic cloning could pave the way for reproductive cloning should be remembered that there is an unbridgeable difference between the two procedures: the implementation or not in a human uterus. It is enough just to ban the implantation in the uterus. If we think that any human cell can theoretically be cloned and generate a new being, we can reach so far as to think that every time we get the cuticle or pluck a hair we are destroying a potential human life. After all, the nucleus of a cell of the cuticle could be put into an enucleated egg, inserted in a uterus and produce a new life!

The practice of therapeutic cloning raises serious questions that require, for the bodies responsible for the study, common sense and ethics, so they do not legalize behavior that is contrary to human interests and on the other hand, an enormous field open to the indiscriminate exploitation.

Stem cells exist in various tissues (such as bone marrow, blood, liver) in children and adults. However, the amount is small and does not yet know in which tissues they are able to differentiate.

The embryonic stem cells are important sources in that it provides promising results in embryonic development in vitro of clones of pet animals clones after the nuclear transfer technique. This is due to the potential application of stem cells in the intrinsic possibility of cellular differentiation in any adult tissue, and in this case in behaving like embryonic cells.

In Brazil the researches, still in progress, indicate that embryonic stem cells would be able to differentiate into almost all human tissues. But the Lei de Bioseguranga (Biosecurity Act)—which generates much controversy—only allows them to be used for scientific purposes and in the case of human, after being stored for more than three years with parental consent.

Advances in biotechnology have meant that the DNA could be manipulated in a controlled manner, generating modified species of rats, mice, sheep, cattle, goats, pigs and even primates. The generation of transgenic models as, for example, the mouse has made possible the use of animals in experiments produced in the laboratory as sources for studying functional characteristics of the man who wish to study.

The advantage of using genetic engineering tools for the development of techniques for production of biopharmaceuticals and vaccines is the possibility of increased income and production capacity, providing a cost reduction as well as the possibility of better process control and quality products. Brazil, today, depends on imports of biopharmaceuticals. The research in the country wants to help change this reality through the development of advanced technologies to obtain recombinant products.

The use of transgenic animals has been shown to be highly efficient for the production of recombinant proteins in milk, at large-scale and lower costs, which have been accepted by regulatory agencies responsible for the production of drugs. Although many ethical issues surrounding the theme appropriate solutions are waited; the transgenic animals are very promising for the biomanufacturing, clinical and diagnostic studies in basic research.

Thus, the expression of biologically active clotting factor IX in expression systems using transgenic animals allowed the evaluation of the functioning of the recombinant expression in transgenic mice (Lisauskas et al., 2008), justifying the applicability of systems in transgenic animals used as bio-factories or bioreactors.

The gene for human coagulation factor IX has been integrated by means of genetic modification by addition, the method used to introduce genes, and in this case, the exogenous gene. Exogenous genes belong to another species, in this case the human species and are used to make an animal to produce a new protein, absent in the recipient plant species, in this case mice.

Since the insertion of the transgene into the host genome by this method as far as we know is random, it can be ineffective or even lethal, due to where the gene is integrated to be uncertain. In the first possibility, the transgene can be inserted in a region of the chromosome that hampers or prevents the expression, so that the animal does not show the phenotype level. In the second possibility, the random insertion can cause, for example, inactivation of a gene critical in embryonic development, resulting in infeasibility or premature death of the animal. In this case, the phenotype of the transgenic animal is independent of the transgene, i.e., was not caused by a characteristic of the inserted gene, but by where the gene was integrated. An important question in experiments with transgenic animals is the “position effect” of genes and the effects of the region in which they occur. That is, we discuss the different patterns of transgene expression in the host genome, linked to its site of integration. Because of these possibilities, the comparison of several transgenic lines with the same genetic modification is necessary if one can infer the non-inclusion of a deleterious gene or trying to correlate it to the level of expression of the transgene insertion site (Lisauskas et al., 2007). In order to apply this model to study events in the generation of elites expression of transgenes, so-called genetic modification directed or controlled (target genes) is the method used for the modification and inactivation of genes that requires knowledge of its location in the genome. This technique allows one to replace a functional gene by a mutated sequence that, once introduced, the original inactivates the endogenous gene, generating an animal model known as knock-out. Likewise, you can change a small gene sequence, generating a knock-in model that will produce a modified protein instead of endogenous protein present in the animal. The term knock-in is because of the endogenous gene of the genome to be removed and replaced by another with a slight modification.

Several techniques are used to produce transgenic animals by adding segments of DNA into the genome—including, pronuclear microinjection of embryos, DNA transfer mediated by sperm, infection of embryos by retroviral vectors, the DNA transfer mediated by transposons, the injection of embryonic genetically modified stem cells, nuclear transfer of genetically modified cells. Among all these procedures, pronuclear microinjection is the most used in the production of transgenic mice. The rate of integration of DNA into the embryonic genome is low—about 1% to 6% (Lisauskas et al., 2008). That is, only a few animals born carry the transgene integrated into their chromosomes. The integration of the transgene for microinjection, as previously mentioned, occurs randomly in the genome, and all animal cells are genetically modified, including the germ ones, so that this change will be transmitted to their descendants. All positive transgenic animal originated from a microinjected embryo is rated as the founder of a single transgenic lineage, which differs from other founders and the insertion site and the number of copies of the transgene in the genome. Another method to be applied in the generation of genetically modified mice is the isolation and maintenance of undifferentiated stem cells in vitro and subsequently to the genetic modification of embryos and injection of these receptors, can produce chimeric mice. This definitely proved that these cells are able to start the process of differentiation and produce a complete individual. The embryonic stem cells are totipotent, i.e. able to generate any kind of tissue. They are derived from embryos at an early stage of development, about four days after fertilization of the egg. The embryo at this stage is called a blastocyst and has hundreds of stem cells in their inner cell mass, which give rise to all tissues of the adult organism. Currently, this technique is routinely applied in mice, one of the only species—besides the human—that the culture of embryonic stem cells is totally dominated. For other species, there is difficulty in maintaining undifferentiated stem cells derived from embryos in culture. In our study, bovine embryonic stem cells were isolated and cultured for long periods while maintaining their differentiation. The next step to the isolation of embryonic stem cells is the selection of those genetically modified, and the technique of choice that proved effective was through laser technique. The transfected cell lineages can form clones and cell banks for future production of transgenic bovines.

There are numerous potential applications of the use of genetically modified animals as models for the study of the causes, progression, stages and symptoms of cardiovascular diseases, autoimmune diseases, neurological disorders among others. By using lineages of genetically modified stem cells may be allowed a detailed analysis of the pathophysiology of diseases, provide also the development of new treatments, new diagnostic tests, effective and inexpensive therapeutic agents more, and long-awaited establishment of protocols to gene therapy.

Moreover, transplantation of organs and tissues from animals into humans, or xenotransplantation, is also the focus of interest in the use of transgenics. There is world-wide shortage of organs for clinical transplantations, and unfortunately many patients die on the waiting list. The advantages of xenotransplantation over traditional transplants include unlimited supply of organs and the consequent reduction of waiting lists. Currently the researchers are developing transgenic pigs whose organs can be safely used for transplantation in humans. Therefore, it is necessary to overcome the immune system that recognizes and destroys all cells that do not have human specific markers on their surface, causing the phenomenon of rejection. To work around this problem, we created transgenic pigs carrying a gene that encodes a protein on the surface of human cells. As a result, these pigs have organs containing human cell markers, which prevent components of the immune system to attack and destroy the receptor organ. Simultaneously, other research groups are studying a different strategy to minimize organ rejection in xenotransplantation. It consists of removing the pig genome, the knockout method, the gene that encodes α-1,3-galactosyltransferase, an enzyme present on the surface of cells from that animal that is recognized by the human immune system. Without this enzyme, the first step in the rejection of the transplanted organ does not happen. Despite the good prospects of xenotransplantation, the use of the method is still the focus of numerous ethical issues that should be fairly discussed.

Another promising application of the transgenics is in the livestock and industry. The methods of selecting desired features such as increasing milk production, are based on selective and conventional breeding animals. However, selective breeding is extremely slow and expensive, besides being a process that does not guarantee the desired results. The new techniques of molecular biology have made possible the introduction or manipulation of desirable features in animals in less time and more accurately. As an example, we mention the generation of transgenic cows, by manipulating endogenous genes, which produce more milk/day.

In the pharmaceutical industry a process called “humanized mouse” is the use of these animal models in developing new drugs. Through this process, a gene is removed from the animal's genome by knock-out technique and, instead, a human gene is inserted by the gene addition method. This technique should greatly facilitate the development of new drugs, reducing costs and decreasing time to reach a new drug to pharmacies. Another important application of transgenic animals is the production of animals known as bioreactors. These pets are usually medium and large, used to produce recombinant human proteins of high biological and commercial interest, such as enzymes, hormones, growth factors, coagulation factors. In general the protein of interest is expressed in the milk of the animal, making production cheaper and more efficient. In 1997, the first transgenic bovine, Rosie the cow, produced enriched milk with the human protein lactalbumin. This transgenic milk is more nutritious for humans than the natural milk, and could be introduced into the infant with a lack of specific nutrients. There is also ongoing research focused on the production of transgenic milk containing the proteins necessary for the treatment of diseases such as phenylketonuria, hereditary emphysema and cystic fibrosis. In our study, transgenic mice were produced in the milk of human clotting factor IX. The recombinant protein generated was tested and proved to be biologically effective in clotting the blood of hemophiliacs.

The future of animal transgenesis is arguably the use of these animals as bio-factories for the production of recombinant proteins for the benefit of our society. This production will provide an unlimited amount of drugs being produced in profusion, and provide them at a low cost and being able to supply banks of hospitals, mainly in the public health. Currently, Brazil spends large amounts of money paying to the importation of drugs not produced by Brazilian industries. With this technology, the country will save investment funds in other poor areas of the country, such as education and public safety. However, despite the advantages of using transgenic animals, Brazil still suffers from many legal and political obstacles that plague the braking of such technology. In a country which currently suffers from a lack of money to import goods essential for the maintenance of quality of life of people with rare diseases, poor distribution of medicines in public health, the constant deaths of people waiting organ to be transplanted, the high cost in the purification of blood products and the risk of possible transmission of these diseases, and the lack of studies of the effect on the application of new drugs handled and distributed commercially, is a crime to attempt to stop the rise of new technology and allow Brazilians in the future, increase their chances of cure or mere expectation of life provided by new coverage of such technology. Based on these aspects, the recombinant DNA technology and genetic engineering that are already available in our country is in line with the realization of the possibilities mentioned above, if influential people in our country tried to give notice about the benefits, the possibilities of long-range technology and real risks in application techniques, instead of working in law and legal forms that meet the real benefits to the population posed by this technology.

This positive revolution in medicine, which involves the production of recombinant effective at low production cost, the unlimited production of organs for xenotransplantation, the manipulation of human stem cells to cure diseases whose only solution is the use of these totipotent cells should be absorbed as the only viable solution to Brazil whose precariousness is the adjective in all main areas of excellence in the country.

In particular, this invention relates to the modification process of bovine embryonic stem cells and purification of proteins generated by such modified cells.

What is clear from the literature, there are no documents suggesting or anticipating the teachings of the present invention, so that the solution proposed here has novelty and inventive step against the prior art.

### OBJECTS OF THE INVENTION

The present invention relates to the modification process of bovine embryonic stem cells, and purification process of proteins generated by such modified cells.

In particular, the present invention lies in the field of medicine and veterinary.

It is therefore an object of the present invention the modification process of bovine embryonic stem cells comprising the steps of:

a) building expression vectors comprising:
a1) at least one nucleotide sequence capable of expressing the human blood clotting factor IX gene and/or its fragments containing bovine signal peptide at the 5′ end;
a2) at least a suitable promoter;

b) contacting bovine embryonic stem cells with the expression vectors from a).

In particular, lineages of bovine embryonic stem cells were isolated from the inner cell mass of bovine embryos produced in vitro in order to become, in the future, sources of bovine embryonic stem cells, to be genetically modified and used for nuclear transfer to obtain transgenic bovines.

It is therefore another object of the present invention the purification process of proteins generated by modified bovine embryonic stem cells comprising the steps of:
a) engage the histidine tail to the 3′ end of at least one nucleotide sequence capable of expressing the human blood clotting factor IX gene and/or its fragments containing bovine signal peptide at the 5′ end;
b) purifying a) promoting the separation of biochemical compounds.

These and other objects of the invention will be immediately appreciated by those who are skilled in the art, and for companies with interests in the segment, and will be described in sufficient detail to be reproduced in the following description.

### DESCRIPTION OF THE PICTURES

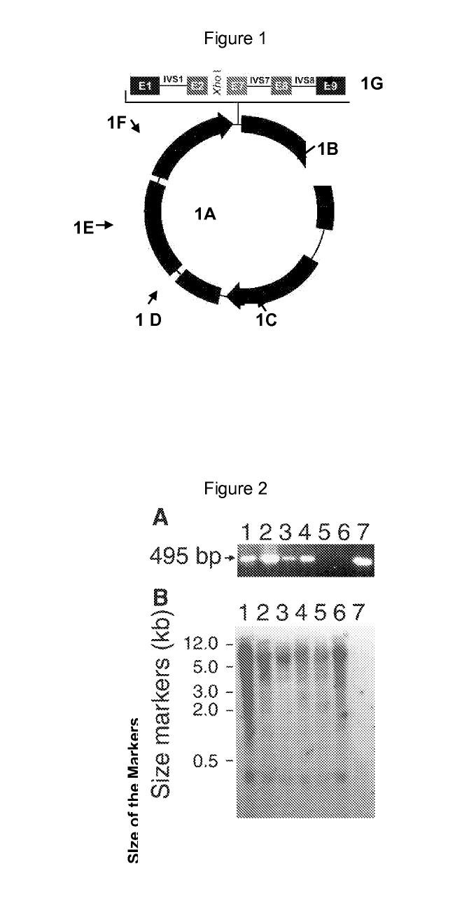

FIG. 1 describes the expression vector pBC1 FIX (1A) used for the generation of transgenic mice. The coding sequence of human FIX gene was fused to the signal of the bovine beta-casein secretion (MAKVLILACLVALALA) and cloned into the XhoI restriction site of the commercial expression vector pBC1 (Invitrogen, USA).

FIG. 1B represents the β-casein 3′ genomic DNA that allows the correct translation and polyadenylation of mRNA.

FIG. 1C represents Ampicillin.

FIG. 1D represents the pMB1 ori.

FIG. 1E represents 2× isolated β-globin.

FIG. 1F (P β-casein) is the induction of transgene expression in epithelial cells of the mammary gland.

FIG. 1G represents fragments of genomic DNA from the coding region of the protein β-casein.

FIG. 2 (A) describes the evaluation by PCR in transgenic mice. 1, 2, 3, 4 correspond to the transgenic mice, five to non-transgenic mice, 6 internal negative control reactions; 7 FIX gene sequence corresponding to 495 bp. FIG. 2 (B) depicts the Southern blot analysis of transgenic lines 1 and 2 (1, 2, 3, 4, 5 and 6) and non-transgenic line (7).

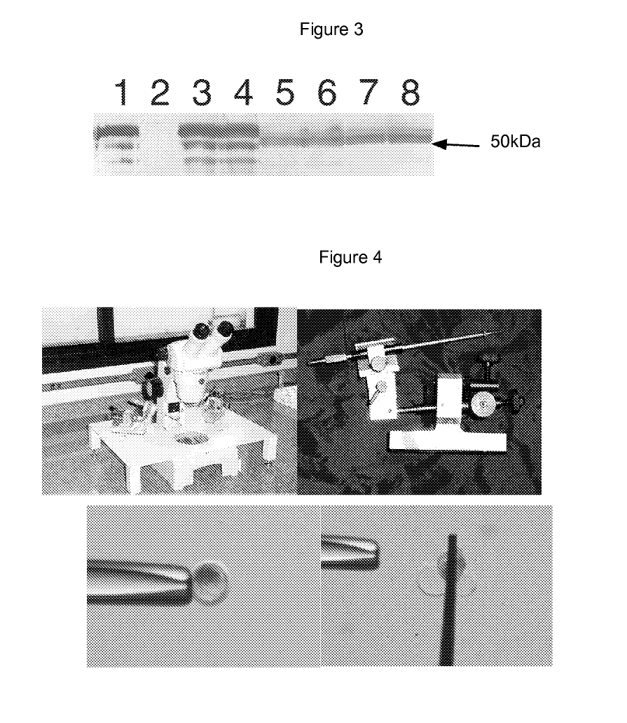

FIG. 3 shows Western blot of the transgenic milk of cows expressing human FIX gene. 1 corresponds to 350 ng of purified FIX protein (Sigma, USA), 2 corresponds to 100 μg of total protein content of milk from non-transgenic female, 3 and 4 to 100 μg of transgenic female founder lineages 1 and 2; 5 and 6 to 100 μg of the F₁ progeny of lineage 1; 7 and 8 to 100 μg of the F₁ progeny of lineage 2.

FIG. 4 describes the manual isolation of embryonic button of bovine embryos produced in vitro.

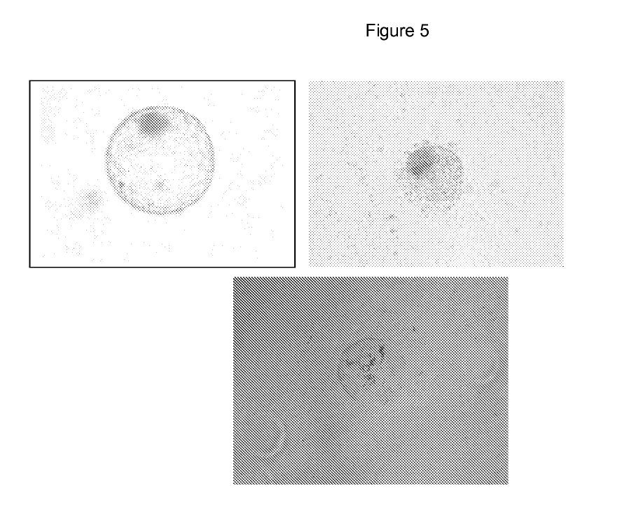

FIG. 5 describes bovines embryos produced in vitro at different stages of development, used for the isolation via laser of its embryo bottom.

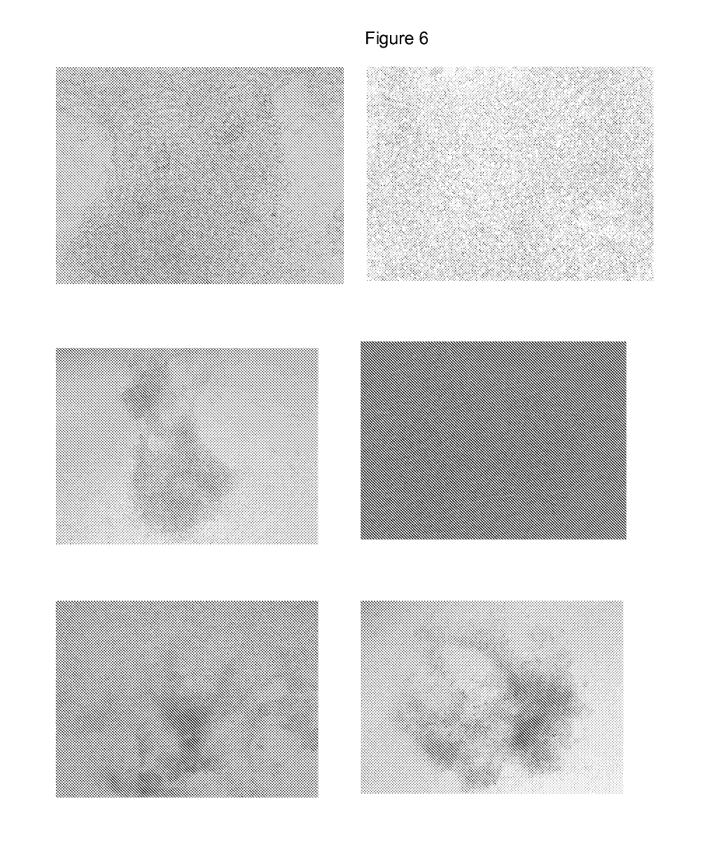

FIG. 6 depicts five (5) Colonies of Bovine Embryonic stem cells isolated by Isolation manual system.

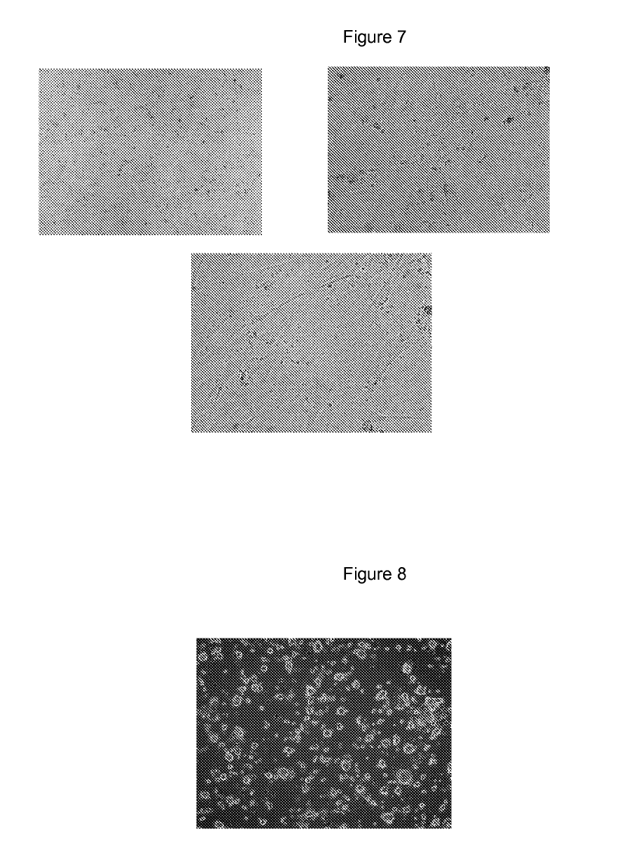

FIG. 7 describes photos at different zooms in the culture of mice fibroblasts used as monolayer basis for growing of embryonic lineages of embryonic stem cells from other species.

FIG. 8 describes of ES cells colonies of transplanted newly picked mice—5×.

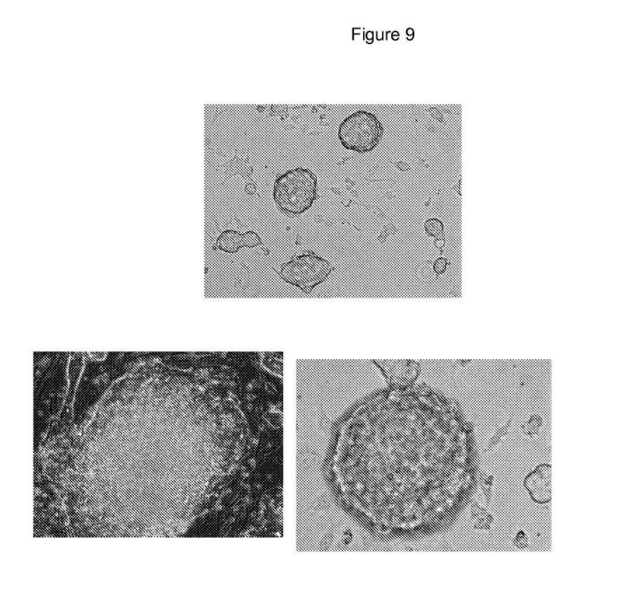

FIG. 9 describes ES cells Colonies newly picked, undifferentiated and at early differentiation

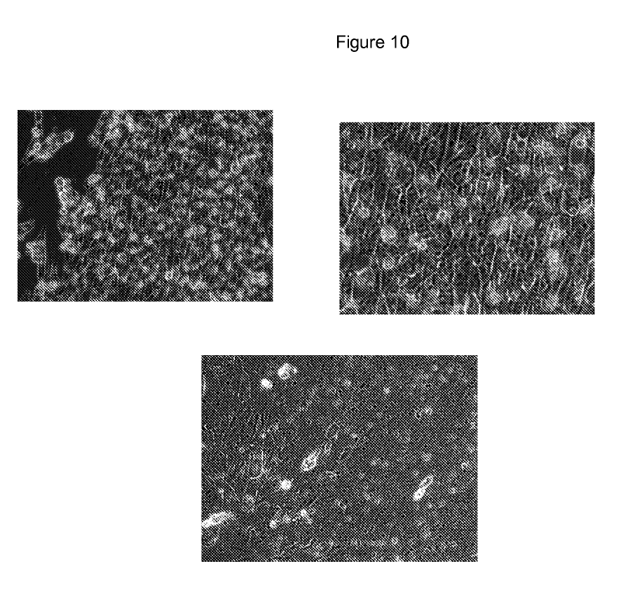

FIG. 10 describes Colonies human ES cells transformed with GFP at different zoom under phase contrast.

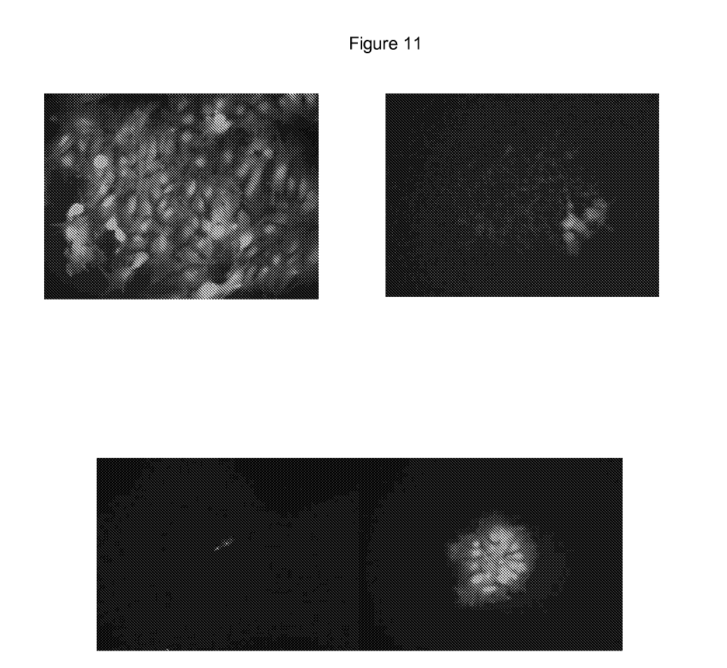

FIG. 11 describes Colonies human ES cells transformed with GFP at different zoom under incidence of Ultra-Violet.

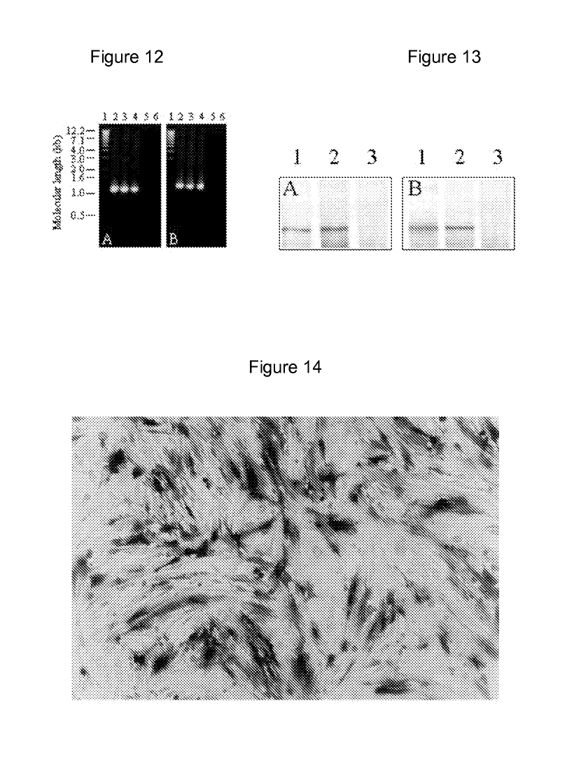

FIGS. 12 and 13 describe RT-PCR and Western blot of BMP-2 and BMP-4, respectively.

FIG. 14 describes fibroblasts transfected with bovine pCMVβ using Lipofectamina (200×).

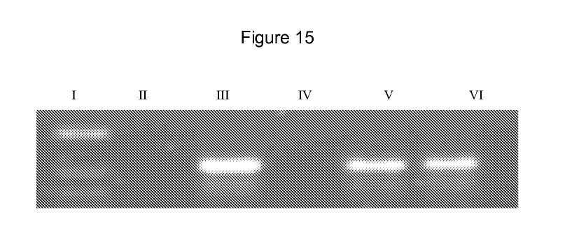

FIG. 15 depicts the detection by PCR of the neo gene in genomic DNA of bovine fibroblasts from two transfected cell colonies, I-molecular weight marker; II-PCR no template to confirm the absence of contamination in the reaction; III-10 pg PCI-neo vector purified IV-200 ng of fibroblasts transfected non-DNA, V-200 ng of DNA from bovine fibroblast transfected and VI-200 ng of DNA transfected cells of the second lineage.

### DETAILED DESCRIPTION

The following examples are not intended to limit the scope of the invention, but rather to illustrate one of many ways to accomplish the invention.

### Modification Process of Bovine Embryonic Stem Cells

The modification process of bovine embryonic stem cells comprising the steps of:

a) building expression vectors comprising:

a1) at least one nucleotide sequence capable of expressing the human blood clotting factor IX gene and/or its fragments containing bovine signal peptide at the 5′ end;

a2) at least a suitable promoter.

b) contacting bovine embryonic stem cells with the expression vectors from a).

### Expression Vectors

It is understood as “expression vectors” vectors capable of expressing the genetic information of a DNA fragment inserted into an exogenous DNA and can be selected from the group comprising plasmids, viruses, DNA plasmids and/or YACs. The expression vectors are coupled to promoters, which are sequences capable of promoting the expression of the DNA in the cell.

### Expression of Recombinant Human Factor IX in the Milk of Mice Construction of Expression Vector

The coding region of human factor IX [(access number to GeneBank XM_{—}010270.4) (Yoshitake et al., 1985) (Text 1)] was amplified by PCR (Polymerase Chain Reaction) from a cDNA library (k Triplex, Clontech, USA) of human liver. These fragments were cloned into pGEM-T Easy vector (Promega, USA) specific for cloning PCR products. The pairs of primers: BCproFIX [5′-GCTCGAGATGGCAAAGGTCCTCATCCTTGCCTGCCTGGTGGCTCTGGCCCTTGCA ACAGTTTTTCTTGATCATGAAA-3′], including the site for the XhoI enzyme (underlined) and the signal sequence of bovine beta-casein secretion (in bold) and FIXstop3C [5′-CCTTCTCGAGCCATCTTTCATTAAGTGAGC-3′], including the site for the XhoI enzyme (underlined), resulting in the amplification of the 1,378 base pairs fragment, the expression cassette to be cloned. PCR reactions were performed in thermocycler tubes in 50 μL reaction containing 10 ng DNA, 60 mM Tris/H₂SO₄ (pH 8.9), 18 mM (NH₄)₂SO₄, 2 mM MgSO₄, 250 nM of each dNTP, 200 nM of each primer, and 5 U of Taq platinum DNA polymerase high fidelity (Invitrogen, USA). The reaction cycles were preliminarily treated at 95° C. for 5 minutes and subjected to 35 amplification cycles consisting of 95° C. for 1 minute, 55° C. for 1 minute and 68° C. for 1 min, with a final elongation cycle of 68° C. for 5 minutes.

PCR products were sequenced using the universal primers M13 and T7 in automatic sequencer ABI Prism1 3700. The 1378 base pairs fragment, from the rearranged factor IX gene was cloned into the XhoI cloning site of the expression commercial vector pBC1 (Invitrogen, USA) to generate the final vector named pBC1 FIX (FIG. 1), which was used in mammalian genetic modifications.

### In Vitro Production of Bovine Embryos

Ovaries from Holstein cows were collected in a refrigerator immediately after the death of animals and transported in DPBS (Invitrogen, USA) in coolers around 37° C. to the laboratory of in vitro production of bovine embryos. 40×12 cm gauge needles were used for vacuum aspiration of the follicles of approximately 10 mmHg (millimeters of mercury).

The oocytes were collected in 50 mL Falcon tubes (Falcon, USA), and when filled were immediately transferred to large Petri dishes (Falcon, USA) to be tracked and evaluated. To the extent that the oocytes were selected, they were placed in small Petri dishes (Falcon, USA) containing the means of selection of oocytes: 200 ml of TL Hepes Solution (Cambrex), 3 mg/mL BSA V (Sigma), 2 mL of PTR [5 mL TL Hepes Solution (Cambrex), 11 mg of Piruvic Acid (SIGMA)], and 100 μL of Gentamicin Sulfate [CAMBREX; [ ] 0.5 μL/mL].

The oocytes classified as grades 1, 2 and 3 (Fish et al., 1997), were placed in a maturation medium containing 4.5 mL of Medium 199 (Cambrex, USA), 50 μl_of NTP, 500 μL of Fetal bovine serum (Invitrogen, USA), 5 μL of gonadotrophin (Sigma, USA), 5 μL of Gentamicin Sulfate (Cambrex, USA) and 5 μl_of estradiol (Sigma, USA) in cell culture incubator at 39° C. and 5% CO₂ in atmosphere with 100% unit.

After maturation of oocytes for 24 hours, these were pre-washed and transferred to plates with appropriate medium for fertilization containing 5 mL of IVF-TL Solution (EmbryoMax, USA), 30 mg of BSA-FAF (SIGMA, USA), 50 μl_of PTR and 2.5 μL Gentamicin Sulfate (Cambrex, USA).

The insemination was carried out with semen of commercial Dutch bull (0.25 mL/frozen straw). The straws were thawed in air for 10 seconds and 20 seconds in a water bath at 37° C. The semen was centrifuged (1000 rpm/6 minutes) in a Percoll gradient medium containing [500 μL of 90% Percoll (Invitrogem, USA) and 500 μL of 45% Percoll (250 μL of 90% Percoll with 250 μL of PTR)]. The supernatant was discarded and the pellet containing the viable sperm was resuspended with 1 mL of TL Hepes medium (Cambrex, USA). The semen was centrifuged again for 6 minutes and the supernatant was discarded around 600 μL, and the remaining 400 μL resuspend the pellet formed.

Immediately prior to insemination, was added to fertilization drops 4 mL of the aliquots mixture of heparin (10 μg/mL, Sigma, USA) and PHE (Penicillamine, Heparin and Epinephrine, SIGMA, USA). The insemination dose at around 0.75 μL/25 oocytes was added to each drop and was incubated at 39° C. and 5% CO2 atmosphere with 100% unit.

The oocytes fertilized for 24 hours were pre-washed and transferred to plates with growth medium containing 5 mL of SOF Solution (EmbryoMax, USA), 2.5 mL of gentamicin Sulfate (Cambrex, USA), 100 μL of PTR, 50 μL of Non-Essential AA Solution (100×) (SIGMA, USA), 100 μL of Essential AA Solution (BME-50×) (SIGMA, USA) and 40 mg of BSA-FAF (Sigma, USA). These embryos were cultured for seven days in an incubator at 39° C. and 5% CO2 atmosphere with 100% unit, before being used in the isolation of its inner cell mass.

### Isolation of Bovine Embryonic Stem Cells and Maintenance of Lineages

A total of 260 embryos after seven days of in vitro production were used for the isolation of bovine embryonic stem cells. The embryonic button or the inner cell mass was isolated by mechanical dissociation and placed in culture dishes on the cell rug formed by PMF P3 cells treated with mitomycin C (Chemicon, USA) and cultured in an incubator at 39° C. and 5% CO2 in the atmosphere with 100% unit.

The growth medium of the colonies of bovine embryonic stem cells called ES cells media (Embrionic Stem cells media) consisted of 300 mL of Knock out D-MEM medium (Invitrogen, USA), 54 mL Fetal bovine serum (Hyclone, USA), 2.4 mL of AA Essential Non-Solution (100×) (SIGMA, USA), 2.4 mL of 200 mM L-Glutamine (Invitrogen, USA), 0.4 mL Gentamycin (Cambrex, USA), 250 μL of β-Mercaptoethanol (Sigma, USA) and 30 μL of leukemia-inhibitory factor (LIF, Sigma, USA).

The renewal of the growth medium was performed every three days, and the rebound was done only every two weeks of growing colonies of bovine embryonic stem cells.

### Description of the Manual Isolation Method of Bovine Embryonic Stem Cells

D-7 embryos were used for the manual removal of stem cells. We used a manual system of micromanipulation where the button embryonic cells were removed precisely by means of ultra-thin razor (Bio-technology, USA) in order to cause less injury to the isolated cells. 05 colonies were obtained by this system stem from isolation that were subjected to molecular analysis for the certification of cellular pluripotency;

The technique consists in choosing embryos Grade I classification by Fish et al. (1997), separate them into individual drops for the removal of the button was embryonal in isolation, avoiding contamination between cell embryos.

Immediately after section, the button embryo was transferred to 04 well plates (Nunc, USA), which means ES cells, the 39° C. and 5% CO2 atmosphere with 100% unit.

### Genetic Modification of Mammalian Cells

The PBC1-FIX vector was used in the stable transformation of clone lineages of somatic cells and bovine embryonic stem cells. Bovine cells were plated on 24 well plates (FALCON, USA) at a concentration of 2×10⁵ cells.

To each well, 0.5 μg DNA (pBC1-FIX) was diluted in 25 μL D-MEM (Invitrogen, USA) without fetal bovine serum (FBS), 4 μL of Plus Reagent (Invitrogen, USA) and a 1 μL lipofectAMINA® (Invitrogen, USA). The solution was homogenized and incubated at room temperature for 30 minutes. The growth medium of plate containing cells (ES cells media) was removed and the mixture containing the expression vector was added to cells. After 5 hours of incubation at 39° C., 5% CO₂ in 100% humidity, the solution was replaced by growing ES cells media with FBS (Invitrogen, USA).

Forty-eight hours after transfection, cells were diluted 1:10 and isolated for maintenance of clone lineages of somatics cells and bovines embryonic stem cells. The lineages of cultured bovine clones were cryopreserved in medium containing 5 mL of ES cells media, 4 ml of fetal bovine serum and 1 mL of DMSO (Dimethyl sulfoxide, Sigma, USA) and stored in tanks of liquid nitrogen to 196 degrees below zero.

### Recovery and Initial Purification of the Protein

Purification of the gene product produced in the milk is made by means of centrifugation in which there is separation of the lipid phase and protein milk. This recovery aims to obtain a high concentration of the purified protein from the initial raw material for the preliminary analysis of bioactivity of the recombinant protein (Lisauskas et al., 2008).

In order to purify the recombinant protein on an industrial scale and commercial, the protein compound must be isolated by HPLC (high performance liquid chromatography) (Van Cott et al. 1996; Wright & Colman, 1997).

### Integration and Analysis of Gene Expression

Molecular analysis [PCR and Southern blot (FIG. 2), western blot (FIG. 3)] and bioassays (Table 1) are conducted to detect the transgenic lines, the expression of recombinant protein in the milk of animals and their coagulant activity.

Western blot analysis confirmed the presence of the correct molecular mass of the recombinant protein (50 kDa) in the milk of transgenic animals, ensuring the correct processing strategy when using the expression vector constructed, based on the use of secretion signal of bovine beta-casein.

### Analysis of Bioactivity of the Recombinant Protein

To prove the activity of the recombinant protein in its action coagulant, the milk of founder animals of the lineage 01, the lineage 02, normal human plasma, hemophilic human plasma, and milk from non-transgenic females were compared. The clotting activity detected in mU/mL (milliunits/milliliters) was 17, 31, 44, 7 and 0, respectively. The clotting time was estimated at 76, 69, 37.6, 87 and 141 seconds respectively. The activity of the protein was estimated as a percentage of their coagulant action (Table 1).

TABLE 1
Clotting activity of recombinant FIX in milk from transgenic and non-transgenic mice.
Bioactivity of the recombinant protein

| | | Milk from | Milk from | | | |
|---|---|---|---|---|---|---|
| | | the founder of | the founder of | Normal | Hemophilic Plasma | Milk from |
| | | transgenic | transgenic | Human | suplemented | non-transgenic |
| | | lineage 01 (R0) | lineage 02 (R0) | Plasma | with FIX (Sigma) | females |
| Western blot | | + | + | + | + | − |
| Clotting | (mU/ml) | 17 | 31 | 44 | 7 | 0 |
| Activity | s | 76 | 69 | 37.6 | 87 | 141 |

Text 1. Genetic Code and polypeptide sequence of the blood
clotting human factor IX gene

Translation of Unknown (1-1320)
Universal code
Total amino acid number: 439, MW = 49445
Max ORF: 1-1317, 439 AA, MW = 49445

| | |
|---|---|
| 1 | ACAGTTTTTCTTGATCATGAAAACGCCAACAAAATTCTGAATCGGCCAAAGAGGTATAAT |
| 1 | T V F L D H E N A N K I L N R P K R Y N |
| 61 | TCAGGTAAATTGGAAGAGTTTGTTCAAGGGAACCTTGAGAGAGAATGTATGGAAGAAAAG |
| 21 | S G K L E E F V Q G N L E R E C M E E K |
| 121 | TGTAGTTTTGAAGAAGCACGAGAAGTTTTTGAAAACACTGAAAGAACAACTGAATTTTGG |
| 41 | C S F E E A R E V F E N T E R T T E F W |
| 181 | AAGCAGTATGTTGATGGAGATCAGTGTGAGTCCAATCCATGTTTAAATGGCGGCAGTTGC |
| 61 | K Q Y V D G D Q C E S N P C L N G G S C |
| 241 | AAGGATGACATTAATTCCTATGAATGTTGGTGTCCCTTTGGATTTGAAGGAAAGAACTGT |
| 81 | K D D I N S Y E C W C P F G F E G K N C |
| 301 | GAATTAGATGTAACATGTAACATTAAGAATGGCAGATGCGAGCAGTTTTGTAAAAATAGT |
| 101 | E L D V T C N I K N G R C E Q F C K N S |
| 361 | GCTGATAACAAGGTGGTTTGCTCCTGTACTGAGGGATATCGACTTGCAGAAAACCAGAAG |
| 121 | A D N K V V C S C T E G Y R L A E N Q K |
| 421 | TCCTGTGAACCAGCAGTGCCATTTCCATGTGGAAGAGTTTCTGTTTCACAAACTTCTAAG |
| 141 | S C E P A V P F P C G R V S V S Q T S K |
| 481 | CTCACCCGTGCTGAGGCTGTTTTTCCTGATGTGGACTATGTAAATTCTACTGAAGCTGAA |
| 161 | L T R A E A V F P D V D Y V N S T E A E |
| 541 | ACCATTTTGGATAACATCACTCAAAGCACCCAATCATTTAATGACTTCACTCGGGTTGTT |
| 181 | T I L D N I T Q S T Q S F N D F T R V V |
| 601 | GGTGGAGAAGATGCCAAACCAGGTCAATTCCCTTGGCAGGTTGTTTTGAATGGTAAAGTT |
| 201 | G G E D A K P G Q F P W Q V V L N G K V |
| 661 | GATGCATTCTGTGGAGGCTCTATCGTTAATGAAAAATGGATTGTAACTGCTGCCCACTGT |
| 221 | D A F C G G S I V N E K W I V T A A H C |
| 721 | GTTGAAACTGGTGTTAAAATTACAGTTGTCGCAGGTGAACATAATATTGAGGAGACAGAA |
| 241 | V E T G V K I T V V A G E H N I E E T E |
| 781 | CATACAGAGCAAAAGCGAAATGTGATTCGAATTATTCCTCACCACAACTACAATGCAGCT |
| 261 | H T E Q K R N V I R I I P H H N Y N A A |
| 841 | ATTAATAAGTACAACCATGACATTGCCCTTCTGGAACTGGACGAACCCTTAGTGCTAAAC |
| 281 | I N K Y N H D I A L L E L D E P L V L N |
| 901 | AGCTACGTTACACCTATTTGCATTGCTGACAAGGAATACACGAACATCTTCCTCAAATTT |
| 301 | S Y V T P I C I A D K E Y T N I F L K F |
| 961 | GGATCTGGCTATGTAAGTGGCTGGGGAAGAGTCTTCCACAAAGGGAGATCAGCTTTAGTT |
| 321 | G S G Y V S G W G R V F H K G R S A L V |
| 1021 | CTTCAGTACCTTAGAGTTCCACTTGTTGACCGAGCCACATGTCTTCGATCTACAAAGTTC |
| 341 | L Q Y L R V P L V D R A T C L R S T K F |
| 1081 | ACCATCTATAACAACATGTTCTGTGCTGGCTTCCATGAAGGAGGTAGAGATTCATGTCAA |
| 361 | T I Y N N M F C A G F H E G G R D S C Q |
| 1141 | GGAGATAGTGGGGGACCCCATGTTACTGAAGTGGAAGGGACCAGTTTCTTAACTGGAATT |
| 381 | G D S G G P H V T E V E G T S F L T G I |
| 1201 | ATTAGTTGGGGTGAAGAGTGTGCAATGAAAGGCAAATATGGAATATATACCAAGGTATCC |
| 401 | I S W G E E C A M K G K Y G I Y T K V S |
| 1261 | CGGTATGTCAACTGGATTAAGGAAAAAACAAAGCTCACTCACCACCACCACCACCACTAA |
| 421 | R Y V N W I K E K T K L T H H H H H H * |

### Transfection of MDBK (Madin Darby Bovine Kidney Cells)

### DNA Preparation

Construction of expression vector was performed according to standard procedures of recombinant DNA technology (Sambrook et al., 1989). The used system of transformation of MSBK somatic cells was standardized according to Oliveira et al. (2005).

MDBK cells were transfected with expression vector called pClneoBeta built in our laboratory, which contains the gene for beta-galactosidase under control of the constitutive CMV promoter and neo gene (neomycin phosphotransferase sequence of codante) under control of the SV40 constitutive promoter. This vector was constructed by inserting the gene for beta-galactosidase after excision of their coding sequence of its origin source vector pCMV-β (7.2 kb) (Clontech, Palo Alto, Calif., USA), with the restriction enzyme NotI and introduced at the site of commercial cloning vector PCI-neo (5.472 bp).

The plasmid was amplified in Escherichia coli DH5α and purified according to Purification kit from Quiagen Giga Plasmid (Qiagen, Valencia, Calif., USA). The concentration of plasmid DNA after purification in Quiagen column was measured by absorbance in the UV wavelength of 260 nm. The purity of plasmid was secured according course in agarose gel at 1% and measuring the A260/A280 ratio in a spectrophotometer. The vector was sequenced and suitable for transformation of cells.

### Cell Culture and Lipofection of MDBK Lineage

Bovine MDBK cell lineages were grown in cell culture bottles (35 cm²) in culture growth RPMI 1640 (Gibco) supplemented with 10% of fetal bovine serum (Gibco), 2 mM L-glutamine, 100 μg/mL penicillin and 100 μg/mL streptomycin at 39° C. in 5% CO₂, 5% O₂ and 93% N₂ in humid atmosphere, being the replacement of the medium held all Mondays, Wednesdays and Fridays. The cells used in the experiments started from the fifth passage of culture, and the passages were performed when cell confluence reached about 80%. The cells were removed from the culture plate treated with 0.05% trypsin-EDTA (1 mL/bottle) for 10 minutes at 39° C. The total number of cells was centrifuged, resuspended and placed back into culture bottles.

The MDBKs were plated at a concentration of 3×10⁵ cells/mL (80-90% confluence) in 24 well plate and transfected in the next day, using the liposome lipofectAMINA® Plus (Gibco, BRL Life Technologies), according to Oliveira et al., 2005.

To each well, 0.5 μg DNA (pClneo-beta) was diluted in 25 μL D-MEM without fetal bovine serum (FBS), 4 μL of Plus Reagent and 1 μL lipofectAMINA®. The solution was homogenized and incubated at room temperature for 30 minutes. The culture growth (RPMI 1640 with FBS) of the plate containing the cells was removed and the solution (without FBS) added to the cells. After 5 hours of incubation, the solution was replaced with culture growth RPMI 1640 with FBS.

### Selection of Transgenic MDBKs Lineages and Isolation of Clone Colonies

48 hours after transfection, the culture growth RPMI 1640 with FBS was replaced with growth medium RPMI 1640 supplemented with FBS and antibiotic geneticina (G418: 400 μg/mL). After 20 days in selective breeding, a single MDBK cell was isolated in an ELISA plate (96 wells) for cultivation of transfected clone lineage under 100 μL of RPMI 1640 with geneticina. In this case, the replacement with new medium was made weekly. When the culture reached the confluence of the well of the ELISA plate, around 15 days of cultivation, the cells were trypsinised and passed to a well of a 24-well plate. Remained in culture until its confluence about 10 days and then were passed to a culture bottle. The entire selection process was conducted in a medium with antibiotic. The colonies were expanded to the entire six bottles of confluent culture, five bottles were used for the isolation of genomic DNA (˜36 μg) and a bottle was reserved for the freezing of cells for the formation of a germplasm bank.

### Detection of the of β-Galactosidase Activity

45 days post-transfection, i.e., before starting to expand in culture bottles, were made tests to detect the expression of β-galactosidase in transfected cells, as security only to expand the transgenic colonies. The cells that expressed β-galactosidase assay were visualized by B-bromo-4-chloro-3-ingylo-β-galactopyranoside (X-gal). The cells were fixed with 50% glutaraldehyde (Sigma, USA) and 37% formaldehyde for 15 minutes and then incubated with X-gal solution (0.2% X-gal, 2 mmol/l MgCl₂, 5 mmol/l K₄Fe(CN)₆, 5 mmol/l K₃Fe(CN)₆). The successful transfection of cells was demonstrated by the blue coloration of colonies clones in its entirety, after 48 hours of incubation.

### Isolation of Genomic DNA

The five bottles of confluent cultures for the isolation of DNA were treated with 0.05% trypsin-EDTA (1 ml/bottle) for 10 minutes at 39° C. The total of all the bottles were centrifuged resuspended in PBS (phosphate buffered saline) and washed three more times in order to remove maximum residue of the medium. Genomic DNA was isolated according McCreath et al. (2000), using a kit of isolation and purification of genomic DNA (Wizard)).

Rescue Technique of Plasmid in E. coli

Ten micrograms of genomic DNA from clone transgenic lineages were digested with appropriate restriction enzyme (which contains only one restriction site within the plasmid, preferably within the gene in question), in this case EcoRI enzyme (Invitrogen, USA) for 3 hours at 37° C. After the reaction, the DNA was precipitated by the addition of 3 volumes of acetate/ethanol, incubated for at least 30 minutes at −20° C. and centrifuged for 15 minutes at 4° C. in refrigerated microcentrifuge (14000 rpm). The DNA was washed with 70% ethanol (v/v) and resuspended in 110 μl of autoclaved MilliQ water. The DNA was linked with T4 DNA ligase (Invitrogen, USA) in 400 μl overnight. The next day, the connection was precipitated (same protocol described) in the final suspension of 10 μl of water. The ligation was transformed into XL1-Blue competent cells by electroporation in BIO RAD system according to the manufacturer's specifications. Plasmid DNA from transformant colonies was extracted using Quiagen kit (“miniprep”) and further analyzed by restriction profile.

### Identification of Transgenes and Flanking Regions

Genomic DNA was extracted from MDBK cells and purified using Wizard® Genomic DNA Purification Kit (Promega, Madison, Wis.). PCR analyses were conducted to confirm the insertion of transgenes.

The nucleotide sequence of the two pairs of used primers were: a) for the Amp^{r} gene: AMP 7 (5′-CTTAATCAGTGAGGCACC-3′) and AMP 850c (5′-TCAACATTTCCGTGTCGC-3′) to amplify 860 bp of the neo gene; b) for the β-galactosidase gene: ER1β-gal (5′-TACGGCCTGTATGTGGTGGATG-3′) e ER2β-gal (5′-CCAGTGCAGGAGCTCGTTATCG-3′) to amplify 820 bp of the gal gene. The cycle conditions were: 96° C./2 minutes; 94° C./30 seconds, 56° C./30 seconds, 72° C./45 seconds; X 40 cycles; +72° C./10 minutes, 4° C./24 hours. PCR products were analyzed by agarose gel in course proving the integration of genes and Amp^{r} e β-gal genes.

The animal's genome regions in which the transgenes were integrated were detected from the same material from the technical rescue of plasmid, and sequenced in automatic sequencer ABI Prism. The sequences were subjected to computer analysis using the GCG molecular biology program package. In this case, primers were designed based on the location of the restriction enzyme whose DNA was initially treated and where probably the animal's genome flanking regions appear. The sequences of primers are: pCINEOB 1013: (5′ 3′) CTCTCCACAGGTGTCCACTC e pCINEOB 1252c (5′ 3′) GTGTCCAGACCAATGCCTCC.

### Integration Analysis

Molecular and biochemical analysis were conducted during the different stages of project development, involving histochemical tests (X-gal), PCR and IPCR. The protocols were performed in accordance with the guidelines of the manufacturer (Stratagene), recombinant DNA technology (Sambrook et al., 1989) and according Aragon et al. (1988).

In this study, we sequenced the flanking integration region of transgenes in 26 MDBK independent cell lineages produced by transfection via liposomes. For the 26 rescued clones, the sequences were in size and quality sufficient to distinguish ambiguity at the insertion site in the bovine genome between lineages. No correlation was established between the site of integration of transgenes and the expression level of beta-galactosidase provided through this site.

PCR analysis confirmed the presence of both transgenes neo and β-gal in all generated clone lineages. Redemptions of plasmids confirmed the presence of part of the integrated transgene and part of the site of integration of the bovine genome (sizes varying from 250 to 930 pb) in the locus of integration. The sequences were compared by BLAST searches of genomic DNA made manually against the GeneBank database using the NCBI web server (http://www.ncbi.nlm.nih.gov).

The results revealed that the transgenes were integrated into 12 different chromosomes, suggesting that there is no preference for chromosomal insertion of exogenous DNA. Similar results were observed for Arabidopsis, in which the insertions were found in five chromosomes (Rios et al., 2002). The presence of the integration of the vector in adjacent regions was observed in two clones, B2 and D8. Two sequences (A1.5 and B2.4) showed no similarity with the bovine sequence available in GeneBank.

Most of the integration events occurred within regions of the genome that are permissive to transcription, as observed by the expression of genes of beta-galactosidase and genes of neomycin. However, all clones in this study were selected under the pressure of positive selection (400 g/ml geneticina), which facilitated the isolation of transfected lineages whose integration occurred in transcribed sequences. Although most transgenes integrate into chromatin regions permissive for transcription, we have not established a connection between integration and the level of transgene expression provided by these sites.

We determine both flanking regions of E4 lineage. This allowed us to compare the locus of the transfected line with the homologous locus in the wild lineage. It was observed that 11 nucleotides were deleted at the left edge of the site of integration of the transgene. This finding reveals an insertional mutation of the ed1 gene, which encodes for the protein Ectodysplasins A. The partial or total deletion of this gene causes the disease known as anhidrotic ectodermal dysplasia in bovines (Drogemuller et al., 2002). In addition, other insertion mutations were observed: in the CL2 clone (RPGR gene, associated with progressive degeneration of the retina due to retinitis pigmentosa), the clone CL1 (in myo10 gene, which encodes myosin X), and in clone C10.3 (in bmp2 gene, which encodes bone-forming morphogenetic 2 protein).

The identification of these types of lineages clones is important for the prevention of undesirable phenotypes for the birth of transgenic animals or for the generation of appropriate models for studies of gene function.

TABLE 2
Obtaining bovine embryonic stem cells
Obtaining bovine embryonic stem cells

| ACTIVITY | RESULTS |
|---|---|
| Growth, replating and freezing of | |
| fedders cells (monolayer cell) | |
| Preparation by growth medium (Mouse | |
| ES cells) | |
| Growth, replating and freezing of | The embryonic stem cells of mice are able to |
| Mouse ES cells | receive such treatment and remain viable after |
| | freezing and replating by at least 5 months of |
| | laboratory work |
| Growth of in vitro bovine embryos and | The font used for the removal of stem cells from |
| beginning of the manual and laser | bovine embryos was D-7 or D-12, produced in |
| training to attempt removal of bovine | vitro (about 25 embryos/week) for the demand |
| embryonic stem cells | for laboratory studies |
| Growth of human stem cells (hESC) | Detection of cells transformed with the gene of |
| transformaded with GFP | interest and isolation based on laser separation. |
| Improved techniques for removal of | Growth of bovine embryonic stem colonies, the |
| bovine embryonic stem cell colonies | establishment of cultures and detection of |
| and the first | molecular markers for both stem cells and to |
| | bovine cells. |

1) Isolation and development of clone colonies from human embryonic stem cells expressing the gene for GFP (Green Fluorescent Protein);

2) Selection via laser isolation and in vitro expansion of elite events of human embryonic stem cells, in the words;

3) Cryopreservation of lineages of transfected human stem cells;

4) The post-thaw growth of lineages of transfected human stem cells for the detection of viability and maintenance of its totipotentiality.

5) in vitro production of bovine embryos as donor sources of embryo button;

6) Isolation of bovine stem cells from the embryo button of embryos;

7) Comparison of two techniques for isolation of bovine embryonic stem cells: isolation and isolation via laser manual;

8) Establishment of lineages of bovine embryonic stem cells suitable for transfection in vitro;

9) Generation of the first bovine cell lineages from the isolation of embryonic stem cells as sources of bovine transgenic nuclei in the generation of genetically modified animals to produce recombinant bioindustrial interest.

### Isolation of Bovine Embryonic Stem Cells

The font used for the removal of stem cells from bovine embryos was D-7 or D-12 produced in vitro.

Two techniques have been established for the removal of stem cells: manual and laser technique.

Manual Isolation (BEM et al. 1987; RUMPF et al., 1992)

D-7 embryos were used for the manual removal of stem cells.

We used a manual system where micromanipulation of the button embryonic cells were precisely removed by means of ultra-thin razor (Bio-technology—USA) in order to cause less damage to cells isolated by this method.

05 stem-colonies were obtained by this isolation system that were subjected to molecular analysis for the certification of pluripotent cell (compatible with stem cell).

The technique consists in choosing Grade I classification embryos (IETS, 2000), separate them one by one into individual drops for the removal of the button to give individual embryo, avoiding cell contamination between embryos.

Once the embryo button is individualized this was cut as precisely as possible, avoiding contamination of the trophoblast cells of the embryo itself. The trophoblast cells inhibits the development of the embryo and the button does not maintain the pluripotency of these.

Immediately after section, the button embryo was transferred to 04 well plates (Nunc, USA), containing ES cells medium (without the addition of LIF reagent) in appropriate incubator. There was cell growth during the 05 months of training, where we observed the first colonies.

Subsequent tests were made to detect molecular markers compatible with the development of bovine embryonic stem cells.

Laser Isolation (P.A.L.M Microlaser Technologies AG, 2003)

D-12 Embryos were used D-12 for manual removal of stem cells.

After the D-7, embryos were transferred to a petri dish suitable for the manipulation via laser containing ES cells medium.

Were cultured for 3 days and incubated in an incubator until the embryos reached the hatching stage cell.

Once hatched, the embryos were fixed to the membrane of the plate used in laser equipment and did not move at the time of incidence of the laser.

We used the system Laser Pressure Catapulting LPC—P.A.L.M Microlaser Technologies (Quick Software Guide MB 2.2-0103 (EN) in which the cells of the embryo are marked by the laser, there is the incidence of the laser cell, the isolation of selected cell group and the suction of this individualized group.

The equipment was set to these standards: first, 40× Lens, Robot LPC; UV Energy slides 37, UV Focus slides 50; occurring laser and focus fits throughout the process and to each new embryo.

The cells removed by this system were isolated every manipulated embryo and placed in 96-well plates. Each week were replating and removed to new wells.

No stem-colonies were obtained by this isolation system due to the idea of setting embryos after the prolonged growth of 12 days have come one month before the end of the internship abroad.

This method is the laboratory procedure to test the effectiveness of this process.

One of the objectives of animal biotechnology is the application of current techniques of genetic engineering for the production of large animals with desirable characteristics. Currently, exogenous genes can be introduced into the genome of mice via the “embryonic stem cells” (ES cells) (Nagy et al., 1990) and by homologous recombination techniques. In this system, cultures of mouse ES cells can be used as a tool for the addition, deletion or silencing of genes at specific sites in the genome. In addition, ES cells have the ability to form stable development of cells derived from three embryonic germ cell layers, and successful differentiation into neurons in vitro in hematopoietic cells and heart muscle.

The isolation of pluripotent ES cells lines from human blastocysts have also been reported (Rhind et al., 2003, Kim et al., 2005). In view of the likely significant contribution to the efforts of ES cells to manipulate the genome, and to replace several injured tissues and organs, it seems appropriate to attempt to adapt this technology for genetic improvement of large animals and to generate transgenic products. Except for mice and humans, sheep (Dattena et al., 2006), pig (Piedrahita et al., 1990), rabbit (Chiang et al., 2007), cattle (Keefer et al. 1994; Verma et al., 2007), rat (Ouhibi et al., 1995) and monkey (Mitalipov et al., 2003) has been reported as potential sources of donor ES cells.

The method used for the isolation of human ES cells has been adapted to other species with a few modifications. However, preliminary attempts to culture the inner cell mass (ICM) of various species under embryonic fedders cells layer of mouse or on primary growth of mouse fibroblasts in the presence of leukemia inhibitory factor (LIF) were rarely successful. A combination of growth factors may be required for the proliferation of pluripotent cells, as demonstrated in cell culture of primary strains of mice (Tielens et al., 2006). In our study, bovine ES cells, isolated from the inner mass of bovine blastocysts produced in vitro was only possible when grown on layer of mouse ES cells, providing in this way growth medium conditioned by such cells in concomitant growth.

But the development of clones of ES cells lineages have the ability to sequences, regulation, or gene expression to be studied, generated from a single cell isolated from specialized tissues and used as donor sources of tested cells.

Based on that, the necessity to develop techniques for isolating cells from tissue under study generated the need to isolate stem cells from bovine embryos produced in vitro, as expected in the use of transfected nuclei for the future generation of transgenic animals used as bioreactors.

A major concern in establishing this technique is to attempt to rule out any possibility of contamination from surrounding cells at the time of their isolation. The contamination is eliminated and the isolated cells can be processed and developed as pure lineages and as potential sources for in vitro transfection.

Once transfected, among the trillions of cells generated as pool in the carpet cell, it can be used to focus the laser to detect only the transfected cells that carpet. The PCR-based procedures are used later for the amplification of sequences of inserted DNA/RNA (Shutz and Lahr, 1998), while methods such as mass spectrometry can be used to determine amino acid sequences of polypeptides that are generated by gene expression generated clone of the lineage (Jimenez et al., 1994).

Besides the potential application of transgenic animal, cellular and molecular biologists have a goal to isolate additional experimental uncontaminated preparations of single cells isolated from tissue culture. Such cells can be examined, manipulated, and potentially cloned in an attempt to provide more biological material for analysis and less complex systems to studies of healthy biological processes and of mutants.

The laser-based system is designed to encapsulate and isolate living cells from living tissue or non-viable cells from regions of fixed tissue. The method involves fixing the tissue regions of interest to a thermoplastic film in which they bond and grow. The film used for microdissection and isolation of cells is attached to a culture plate and specially treated to absorb the laser light. Thus, cells/individual groups of cells in tissue regions that are chosen to be catapulted are isolated (Feng et al., 2000), and therefore placed in growth medium for the development of specific lineages clones (Stich et al. 2003).

The laser technique provides the opportunity to use single cells isolated from tumor tissue and healthy living for functional analysis or preparation of clone libraries for various investigations in cell biology, pharmacology, drug development, environmental testing, and development of customized vaccines against specific tumors.

The selection, isolation and analysis of living cells of tissues expressing chimeras of green fluorescent protein (GFP) and subsequent analysis of the expression or function of cells transformed with GFP are also possible applications. In this study, human embryonic stem cells transfected with GFP were used to test the isolation of individual cells via the application of laser and cell growth viability after isolation. Colony growth clones of human embryonic stem cells was possible by using cells isolated via laser and growing them on monolayer cells of fedders cell of mice.

Based on this, the isolation technique via laser and via manual opens a perspective for the future generation of viable clone lineages of stem cells for studies of gene function, gene expression and the generation of nuclei transformed with exogenous genes, mainly of pharmaceutical interest as sources of donor nuclei for nuclear transfer and the generation of animal bioreactors.

### Growing of Fedders Cells (Monolayer Cell)

The monolayer cell of Fedders cells (mouse fibroblasts) is crucial to prepare the conditioning of cell growth medium prior to addition of stem cells, both human and bovine embryos. This allows the adhesion monolayer colonies of stem cells, and the conditioning of growth medium in which the stem cells will be received.

The growth medium for Fedders is called MEF cells.

TABLE 3
Growth Medium: MEF

| | | |
|---|---|---|
| D-MEM | 405 | mL |
| FCS (Fetal Calf Serum) | 75 | mL |
| L-Glutamine | 5 | mL |
| NEAA | 5 | mL |
| Gentamycin | 5 | mL |
| HEPES | 5 | mL |
| Total | 500 | mL |

For the cultivation of Fedders cells (monolayer) were used five Petri dishes previously treated with “Ultrapure water with 0.1% gelatin” (filtered and conditioned to ambient T ° C.). Was added 10 mL of the above solution and left for 30 minutes for the effective treatment of the plates that received Fedders cells.

A aliquot of PMEF-P3 cells was thawed (resistant neomycin) for the cultivation of five Petri dishes. It was centrifuged and resuspended in 5 ml of MEF growth medium to Fedders cells and was added 1 mL of medium+cells in each petri dish treated by supplementing with additional 4 mL of medium/plate.

Plates were incubated for about 24 hours before the addition of Mouse Embryonic Stem Cells.

For the maintenance of feeder cells it should replate them every 2 or 3 days of culture when they reach confluence.

### Replate of Embryonic Stem Cells—ES Cells

When the confluence of the plates containing Fedders cells+stem cells is reached about every 3 days of cell culture, it is necessary to renew cells and reduced cell density in each culture dish in order to avoid death by apoptosis due the high concentration of cells.

To perform the replate of human stem cells from mice and embryonic bovines it was used the protocol as follows:
Pre-rinsing the plates with 5 mL of PBS;
Add 5 ml of Trypisin-EDTA in the plate containing ES cells;
Waited for 10 minutes at 37° C. (within the incubator);
Gathering the ES cells+Fedders cells+Trypsin in a 15 mL Falcon tube;
Add 5 mL of ES media in the tube in order to paralyze the action of trypsin;
Centrifuge for 4 minutes at 1000 rpm;
Neglecting the supernatant and resuspended pellet with 10 mL of ES media, well homogenized to obtain single cells;
Dilute 1:4 to the cells;
Replacing the growth medium of the plates of feeders by adding 5 mL of ES media+ES cells;
Added another 5 mL of ES media on each plate;
Was cultivated at 37° C., 5% CO₂ with high humidity.

### 1× Freezing Media (Method of Freezing Cells)

Each replate is required to cell cryopreservation of cultured cells in order to avoid any risk of subsequent contamination of culture and the loss of cell passage in which the cells were.

The identification of each passage is important for feasibility studies in cell culture, since these cells can be used as donors of nuclei using a transgenic nuclear transfer.

The means of freezing the cells and the protocol are described below:

| | |
|---|---|
| ES media + LIF | 5 mL |
| FCS (Fetal Calf Serum) | 4 mL |
| DMSO | 1 mL |
| Total | 10 mL |

Pre-rinsing the plates with 5 mL of PBS;
Add 5 ml of Trypisin-EDTA plate containing ES cells;
Waited for 10 minutes at 37° C. (within the incubator);
Gathering the ES cells+Fedders cells+Trypsin in a 15 mL Falcon tube;
Dissociated to the colonies in order to freeze them as single cells;
Add 5 mL of ES media in the tube in order to paralyze the action of trypsin;
Centrifuge for 4 minutes at 1000 rpm;
Neglecting the supernatant and resuspended the pellet with 2 mL of 1× media Freezing pre-cooled;
Immediately, transferred 1 mL of suspension for each vial of freeze;
Moved the vials to a box with pre-cooled isopropanol inside at −80° C.;
After 24 or 48 hours, the vials are transferred to the canister of liquid nitrogen.

### Means of Embryonic Stem Cells of Mice: Knock Out mES Media

In order to cultivate embryonic stem cells from mice and bovines, the growth medium was prepared according to the protocol below:

| | | |
|---|---|---|
| Knock out D-MEM | 300 | mL |
| FCS (Fetal Calf Serum) | 54 | mL |
| NEAA | 2.4 | mL |
| L-Glutamine | 2.4 | mL |
| Gentamycin | 0.4 | mL |
| B-Mercaptoethanol | 250 | μL |
| LIF | 30 | μL |
| Total | 300 | mL |

*Homogenizer and filter 0.22μ

The based growing medium is prepared and stored at 4° C.

It should filter the medium after doing it and repackage it for up to 2 weeks.

Reagent LIF is used in addition to the monolayer (Fedders) for maintenance of cell totipotency.

Human Embryonic Stem Cells, hESC—Culture Media

In order to cultivate human embryonic stem cells, the growth medium was prepared according to the protocol below:

| | | |
|---|---|---|
| D-MEM F12 Media | 200 | mL |
| Knockout Serum Replacer | 50 | mL |
| L-Glutamine (200 mM) | 1.25 | mL |
| NEAA (100X solution) | 2.5 | mL |
| Gentamycin | 0.4 | mL |
| B-2-Mercaptoethanol (BME) | 0.85 | μL |
| B-FGF stock solution (in the freezer) | 0.5 | mL |

* Homogenizer and filter 0.22μ

B-FGF Stock solution (Basic Fibroblast Growth Factor—bFGF, human recombinant, Invitrogen, USA)

Prepare 0.1% BSA in DPBS:
0.3 g BSA in a 50 mL Falcon tube.
Was added 30 ml of DPBS and was well mixed.

Was separated 5 mL from 0.1% BSA in DPBS:
Tube B-FGF (10 mg) and mix up.

Was aliquoted 500 mL in each eppendorf and identified it;

Put up to −20° C.

Cultivation of human embryonic stem cells modified with green fluorescent protein (GFP) with Green Fluorescent Protein hESC (ATCC—PubMed: 12529550)

The hESC medium was conditioned on MEF media for 24 hours to be used on plates containing the colonies GFPhESC.

Every 24 hours the MEF medium was collected (18 mL), added B-FGF (140 mL), filtered and added to culture plates containing cells GFPhESC.

### Isolation of GFPhESC Cells Via Laser

1. Detection of human embryonic stem cells carpet transformed with GFP, based on the fluorescence of cells after stimulation with Ultra-violet and green display filter;
2. Immediately after the detection of potentially modified cells, we adjusted the laser equipment in the following specifications: 10× Objective, Robot LPC; UV Energy slides 46, UV Focus slides 73;
3. Laser was applied to the cells and collected in the “cap” (small amount of growth medium for the packaging of only one cell) of the equipment;
4. Immediately after harvesting, the cells were placed in ELISA plates for growth of isolated human colonies with GFP.

### Cultivation of Bovine Embryos In Vitro

### (Greve et al. 1991; Krimpenfort et al., 1991, Carolan et al. 1992; Bols et al., 1995, Fish et al. 1997, Rumpf et al., 2000)

Cultivation in vitro of bovine embryos was performed at the Laboratory under the responsibility of Dr. Rick Monson and supervision of Dr. Dave.

The protocol used was developed by researchers at the laboratory, based on several bibliographies on the subject. Because of this, the protocol was not allowed to describe its components.

It produced a total of 260 embryos as shown below:

TABLE 3
Production of embryos in vitro as sources of embryo bottom
for the removal of bovine embryonic stem cells.

| Date | Oocyte/cleavage | D7 Blastocysts | D8 Blastocyst |
|---|---|---|---|
| 5/Set | 76 | 11 | 7 |
| 12/Set | 58 | 12 | 5 |
| 19/Set | 59 | 12 | 9 |
| 2/Out | 83 | 14 | 10 |
| 9/Out | 38 | 0 | 3 |
| 16/Out | 56 | — | 15 |
| 23/Out | 77 | 7 | 5 |
| 30/Out | 131 | 10 | 11 |
| 6/Nov | 154 | 7 | 17 |
| 13/Nov | 144 | — | 30 |
| 20/Nov | 105 | 0 | 9 |
| 27/Nov | 119 | 11 | 19 |
| 4/Dez | 124 | 9 | 17 |
| 11/Dez | 61 | 10 | — |

| | | |
|---|---|---|
| TOTAL | 1285 | 260 embryos |

### FIV Procedures

### Day −2: Follicular Aspiration

1. The ovaries were collected in a refrigerator, and transported in coolers around 30° C., using air transport;

2. In the laboratory, were washed in tap water and heated to about 35° C. and ready to be vacuumed;

3. Gauge needles were used for the 40×12 vacuum aspiration of the follicles of approximately 10 mmHg;

4. The oocytes were collected in 50 mL Falcon tubes, and when filled, were immediately transferred to large Petri dishes to be tracked and evaluated;

5. As the oocytes were selected, were placed in small petri dishes containing the oocytes through selection of appropriate protocol (not authorized to be disclosed).

### DAY −1: Maturation of Oocytes

1. After being selected, the oocytes of grades 1, 2 and 3 were placed in plates with drops of appropriate growth medium for oocyte maturation Protocol (unauthorized);

2. They stayed for 18 to 24 hours in incubators appropriated for maturation.

Day 0: Fertilization of Matured Oocytes

1. The oocytes matured for 24 hours were pre-washed (to remove any residual of growth medium maturity) and were transferred to other plates with drops of growth medium suitable for oocyte fertilization Protocol (unauthorized);

2. The semen was prepared by specific gradient for the separation of viable sperm for fertilization of oocytes;

3. The drops were placed inseminated 24 hours in an incubator suitable for fertilization;

### Day 1 to Day 7: Cultivation of Embryos

1. The oocytes fertilized for 24 hours were pre-washed (to remove any residual of growth medium maturity) and were transferred to other plates with drops of growth medium suitable for oocyte fertilization Protocol (unauthorized);

2. The embryos were cultured for 7 days in an incubator suitable for the embryonic development;

3. On Day 7, one of the embryos was delivered to the Stem Cells laboratory, under the supervision of Dr. Gabriela Cezar, to attempt to manually isolate bovine embryonic stem cells;

4. The other part was cultivated until Day 10 in growth medium suitable for ES cells, in which embryos then were used to isolate via laser bovine embryonic stem cells.

Construction of expression vector was performed according to standard procedures of recombinant DNA technology (Sambrook et al., 1989). The system of transformation of somatic cells was standardized according to Oliveira et al. (2005) and applied to the bovine fibroblasts (paper 5).

The commercial expression vector pCI-neo (Promega, Madison, Wis., USA) was used for stable transfection of bovine fibroblasts. This vector carries the neomycin phosphotransferase gene under control of the constitutive CMV promoter and poly-A sequence.

The plasmid was amplified in Escherichia coli DH5α and purified according to Purification kit from Quiagen Giga Plasmid (Qiagen, Valencia, Calif., USA). The concentration of plasmid DNA after purification in Quiagen column was measured by absorbance in the UV wavelength of 260 nm. The purity of plasmid was secured according course in agarose gel at 1% and measuring the A260/A280 ratio in a spectrophotometer.

### Gerneration of Transgenics Bovines

The steps of: a) obtaining lineages of bovines fibroblasts derived from animals with milk aptitude suitable for transfection in vitro; b) Transfection and isolation of donor of clone lineages; c) Selection of elite events; were conducted according to Oliveira et al. 2005.

The steps of: d) Preparation of donor cells; e) Cytoplasm receiver (oocytes collected in vivo and in vitro) of animals of dairy breeds; f) Enucleation (micromanipulator); Transfer of transgenic donor cell to the cytoplasm and fusion receptor (by electric stimuli); g) Artificial activation in vitro embryo culture; h) development evaluations (cleavage rate and blastocyst rate); i) Transfer of embryos into recipient cows; are described in the paper 5 for the generation of transgenic bovines using fibroblasts genetically modified as transgenic donor sources of transgenic nuclei by nuclear transfer technique.

### Preparation of Donor Cells

Transfected fibroblast cells were plated at a concentration of 0.5×10⁵ cells in 24-well plates and grown in FAEX+10% fetal bovine serum growth medium until reaching 100% confluence. After that, the growth medium was replaced by the medium with low level of serum (5%) and the cells incubated at 38° C., 5% CO₂ for 4-8 days until nuclear transfer. Immediately before nuclear transfer, donor cells were collected by trypsinization using 0.05% trypsin-EDTA, washed twice, and resuspended in medium containing 1% bovine serum albumin (BSA).

### Cytoplasm Receiver

The oocytes that were used were from live cows and slaughterhouse ovaries. In the first case were collected by follicular puncture guided by ultrasound of separate cows for this purpose, while follicles of 2 to 8 mm were punctured from ovaries from a slaughterhouse for a vacuum system. The cumulus-oocyte complexes (COC's) with homogeneous cytoplasm and more than 5 layers of granulosa cells were selected. These were separate groups of twenty to thirty that were placed in wells containing 400 μl of in vidro maturation medium (IVM) covered with mineral oil and incubated at 39° C., 5% CO₂ atmosphere with saturated humidity. The maturation medium (IVM) consisted of TCM 199 supplemented with 10% v:v fetal bovine serum (FBS), 0.1 U/ml of FSH, 0.1 U/ml of LH, 0.1 mg/ml of L-glutamine and 1% of penicillin/streptomycin. After 22 to 24 hours of onset of maturation, the COC's were denuded by pipetting in a solution of 0.2% hyaluronidase.

### Enucleation

Before heading to the enucleation, the oocytes were placed in the medium of the bench plus Hoechst 33342 vital dye and cytochalasin D for 10 to 15 minutes. The medium bench consisted of Hepes TCM 199 with the addition of 10% v:v FBS and 1% penicillin/streptomycin.

The oocytes were manipulated in medium supplemented bench with cytochalasin D.

To verify the removal of the metaphase plate and first polar body, the sucked biopsy through a pipette 25 to 30 μm in diameter was exposed to ultraviolet (UV). The chromatin stained with Hoechst 33342 when exposed to UV fluoresces in the light, demonstrating the correct procedure.

### Transfer of the Donor Cell and Fusion

The transfected fibroblasts were introduced into the perivitelline space of enucleated oocyte. Then the complex fibroblast-cytoplasm were aligned between two electrodes and subjected to two DC pulses (direct current) of 1.5 kV/cm for 50 pseg. These electrical stimuli were applied in solution to 0.28 M mannitol, 0.1 mM MgSO₄ and 0.05 mM CaCl₂ in order to promote temporary formation of pores in the plasma membranes, thus inducing the fusion of two structures. After electrofusion the reconstructed embryos underwent bath bench in the medium and later were placed in growth medium.

### Artificial Activation and Embryo Culture

Three to five hours after application of electrical pulses has led to activation of complex-fibroblast cytoplasm. These were incubated in solution 5 μM of ionomycin for 4 to 5 minutes or in a solution of 7% ethanol for 5 minutes. Then, the structures have undergone to SOFaa medium baths (Synthetic oviduct fluid) and was assessed the rate of fusion before being transferred to the culture plate. The in vitro culture (IVC) of reconstructed embryos was performed in wells containing 400 μl of SOFaa medium supplemented 5% v:v FBS, 0.5 mg/ml of myo-inositol, 0.1 mg/ml citrate tri-sodium, 0.1 mg/ml L-glutamine, 20 essential amino acids and seven non-essential. The SOFaa medium contained a layer of 400 μl of mineral oil covering it. The MIC was performed at 39° C., 5% of Cos, 5% O₂ and saturated humidity.

### Development Reviews of Development and Transfer of Embryos

48 hours after electrofusion, the cleavage rate of embryos was evaluated. The in vitro growth followed by 7 to 8 days after the merger, when it was found the rate of blastocyst. The embryos were transferred by non-surgical method for recipients cows who had uterine age synchronized with the embryo one. These cows were kept under observation for the return of heat and had the pregnancy confirmed by ultrasound at 30 to 35 days of gestation.

PCR and Southern blot were conducted to detect the stable integration of the transgene in both the donor cells and cells of the transgenic fetuses.

Our results showed that transfection of somatic cell of transgenic nuclei donors does not negatively influence the rates of production efficiency in vitro embryo after the process of TN (fusion, cleavage and blastocyst percentage), as concluded by Roh et al. (2000) and Han et al. (2001) for bovines. Likewise, published data from transfected pig fetal fibroblasts with the gene EGFP (enhanced green fluorescent protein) (Koo et al. 2001; Martinez Diaz et al., 2003) corroborate our findings. However, similar results have been contradictory in bovines, even with data from the same research group. Arat et al. in 2001 reported similar efficiencies between granulosa cells of transfected and non-transfected adult animals in their cleavage rates and blastocyst rates. Even though, Arat et al. in 2002 showed that adult and fetal cell lineages expressing EGFP had capacity for in vitro development of after lower TN when compared with the same transfected cell lines, but not expressing EGFP. More examples and contradictory results have been observed in bovines, some articles (Zakhartchenko et al. 2001; Bhuiyan et al., 2004) indicate a significant decrease in blastocyst rates when used transfected cells compared with non-transfected cells, while others authors reported no such differences (Brink et al. 2000; Brophy et al., 2003). Zakhartchenko et al. (2001) attributed the low rates of pos-reconstruction embryonic development with transfected fetal fibroblasts to long periods of culture required for transfection and selection of elite events, but not to the transgene itself.

Probably, the differences between reported results so far are due precisely to the type of expression vector, cell transfection protocols (Bhuiyan et al., 2004), methods of TN and culture conditions of donor cells from their transgenic nuclei (Wells et al. 2003; Miyoshi et al., 2003). In addition, the site of integration of the transgene used and its possible interference in the expression of endogenous genes may influence the results (Hodges and Stice, 2003).

In our study there were no significant differences in rates of early pregnancy (<than 40 days of gestation) between transfected and non-transfected donor nuclei cells, also reported by Zakhartchenko et al. (2001). On the other hand, Forsberg et al. (2002) reported lower rates of pregnancy post-TN for transfected donor cells than for non-transgenic cells. In our work, the small number of receptors animals gestating transgenic and non-transgenic cloned embryos precludes conclusive results about the survival of transgenic or non-transgenic embryos. The decrease in pregnancy rates found in transfected embryos may be partly explained by the increased incidence of apoptosis in blastocysts produced from long periods of cultivation of the nuclei of donor cells for NT as well as reported by Jang et al. (2004). These authors also reported that the development of in vitro parameters (rates of blastocyst and blastocyst cell count) are not affected by prolonged periods of cell cultures.

The fibroblast lineages used in our study were isolated from ear skin of an embryo clone calf (Sousa et al, 2000; Iguma et al., 2001). This cell lineage was chosen because of superior results in pre-experiments for this purpose. Among these, karyotyping (data not shown) and high transfection efficiency (60-70%; Oliveira et al., 2005).

For the purpose of the cloning of transgenic animals, the use of adult somatic cells is more advisable than the use of fetal cells, especially when the choice allows you to select animals with high production features (high milk production and high growth rate), valued genetic meritand and assured animal sanity.

Once the expression vector pCI-neo used has a single restriction site for BamHI, Southern blot analysis of transgenic fetuses were performed in order to confirm the integration of the transgene, and also highlights the presence of only one integrated copy in the bovine genomee. Both fetuses showed the same pattern of integration, since they were generated from the same transfected cell lineage.

In short, the work did not find significant differences between the rates of embryonic development after TN, when compared transfected and non-transfected nuclei donor lineages. In other words, the results showed no deleterious effects when using the technique of cell transfection, to the gestational age of 40 days in the transgenic fetuses were extirpated for molecular analysis of the transgene.

### BMP-2 and BMP-4

cDNA of bone morphogenetic protein-2 and bone morphogenetic protein-4 (BMP-4) genes were synthesized from total RNA extracted from bone tissue of patients with facial trauma (fractures of the jaw between the 7th and 10th days post-trauma) and cloned in a vector for expression in mammalian cells, under control of the promoter of cytomegalovirus (CMV). The vectors containing the genes BMP-2 and BMP-4 were used for transfection of bovine fibroblasts. mRNAs were indirectly detected by RT-PCR in transfected cells. The proteins BMP-2 and BMP-4 were detected by Western blot analysis. The results demonstrate the possibility of production of these cell growth factors in bovine fibroblasts. These cells may be used as sources of donor genetic material for the nuclear transfer technique to generate transgenic animals.

Total RNA was isolated from the bone tissue of these patients. RNA was isolated using Trizol and cDNA synthesis was performed with the Superscript II kit, according to the manufacturer's instructions.

The BMP genes were amplified by PCR using primers for BMP-2 (F=GTGCTTCTTAGACGGACTGC e R=GTACTAGCGACACCCACAAC) in order to amplify the 1.233 pb of BMP-2 gene and BMP-4 (F=AGCCATTCCGTAGTGCCATC e R=AAGGACTGCCTGATCTCAGC) to amplify the 1.373 pb of BMP-4 gene.

Each PCR reaction was performed in 25 μl mixtures containing 60 mm Tris-504 (pH 8.9), 18 mM ammonium sulfate, 2 mM of MgSO₄, 200 mM of each dNTP, 200 mM of each primer, 1 U high-fidelity platinum Taq polymerase. The mixture was deposited on mineral oil, denatured for 2 min at 94° C. in a thermal cycler MJ Research and amplified by 30 cycles (94° C. for 15 s, 55° C. for 30 s, 68° C. for 2 min). The products were run on 1% agarose gel, labeled with ethidium bromide and visualized in UV light. The amplified DNA sequences were cloned with pGEM-T vector and sequenced. The genes were then cloned into the NotI restriction site in the pCMV-B vector, replacing the B-galactosidase gene under control of the cytomegalovirus (CMV) promoter for expression in mammalian cells to generate pCMVBMP2 and pCMV-BMP4 vectors.

Bovine fibroblasts are transfected with pCMV-BMP2 and pCMV-BMP4 vectors using LipofectAmine Plus, according to the manufacturer's instructions. In order to detect the gene expression of BMP-2 and BMP-4 (mRNA) in cells of transgenic fibroblasts, RT-PCR was performed. The isolation of total RNA and PCR were performed as previously described. RT-PCR amplification of BMP-2 and BMP-4 were performed in three independent lineages of fibroblasts. RT-PCR with total RNA without DNA synthesis showed no amplification, confirming the absence of contaminants in the sample. Western blot was performed as previously described, using monoclonal antibodies, which revealed the production of BMP-2 and BMP-4 proteins.

RT-PCR analysis of bovine fibroblasts transfected with vectors pCMV-BMP2 (A) and pCMV-BMP4 (B). Row 1:1 kb ladder; Rows 2-4: cell lineages of transfected fibroblasts; Row 5: cell lineages of non-transfected fibroblasts; Row 6: total RNA without DNA synthesis.

Western blot analysis showing the presence of proteins BMP-2 and BMP-4 in transfected bovine fibroblasts.

### Liposomes for Fibroblasts Transfection

Development of efficient transfection system in cells using liposomes, particularly in fibroblasts, caprines, ovines and bovines, establishing a foundation for use of transfected fibroblast cells to generate transgenic animals through nuclear transfer technology and gene function studies.

### Fibroblast Cell Culture

Biopsy of the skin of the ear of bovines, caprines and ovines.

Samples washed 3× with 1 ml of cold PBS supplemented with penicillin (100 IU/ml) and streptomycin (100 μg/ml). The skin fragments were washed 4× with 1 ml of trypsin (trypsin 0.05% w/v EDTA 0.02% w/v) and transferred to cell culture bottles containing 4 ml of D-MEM, 10% fetal serum, penicillin and streptomycin. Cells were grown at 37° C. and 5% CO₂ until they reached 70% confluence. Cells were grown tripnized and transferred to new bottles for cell culture without the original fragments of skin.

### Vectors

pCMVB was used, vector that carries the gene for b-galactosidase under the control of CMV promoter sequence and a poly-A sequence.

It also used the pCI-neo vector to allow stable transfections, which carries the neomycin phosphotransferase gene under control of CMV promoter sequence and a poly-A sequence.

Plasmids were amplified in DH5α strain of E. coli and purified using Qiagen Giga Plasmid Kit. DNA plasmid concentrations were measured by UV 260 nm absorption and purity of plasmid DNA was assessed using electrophoresis with agarose gel and A260/A280 ratios.

### Parameters Affecting Transient Transfections of Bovines, Caprines and Ovines Fibroblast Cells

Several parameters were measured as: 1) cell transfection efficiency of DNA/liposome, 2) Effect of cell density in the rate of transfection using bovine fibroblast cultures, 3) Effect of vector plasmid transfection efficiency in bovine fibroblast cells.

### Biochemical Assay of β-Galactosidase

Expression of β-galactosidase was detected 24 hours after transfection and is calculated under an optical microscope (100×).

### Stable Transfection of Fibroblasts

Bovine fibroblast cells were grown in culture dishes and transfected with 0.5 g Lipofectamina and PCI-neo (8.3 mL/ml) and Plus reagent (13.3 mg/ml). 48 hours after transfection, cells were diluted 1:10 and G418 was added to reach final concentration of 0.5 mg/ml. Transfected cells reached subconfluent after 7 days of selection. In the third passage, a portion of cells was diluted 1:10, grown under selective pressure of antibiotics and cryopreserved. The other portion was diluted 1:1000 and grown under selective pressure of antibiotics during seven days. Individual colonies were isolated and expanded for cryopreservation, counting chromosomes and PCR analysis.

### PCR Analysis

Genomic DNA of two lineages of transfected and non-transfected bovine fibroblasts were extracted and purified. Genomic DNA was used as template in PCR reactions containing primers specific to the neo gene (420 pb): NPT151 (5′-ATGATTGAAGAAGATGGATTG-3′) e NPT941(5′-GAAGAACTCGTCAAAGAAGGCC-3′). The PCR was performed in a final volume of 25 μL containing 10 mM Tris-HCl, pH 8.4, 50 mM KCl, 2 mM MgCl₂, 160 mM of each dNTP, 0.4 mM of each primer and 2 U Taq polymerase. The PCR comprised 35 cycles (940/1 min; 600/1 min, 720/1 min) in PT-100 thermal cycler. Amplified PCR products were analyzed on 1.5% agarose gel with ethidium bromide (50 mg/mL) and the images were scanned.

Those skilled in the art will value the knowledge here, and may reproduce the invention in the manner shown and other variations which fall within the scope of the appended claims.

1. A process for the modification of bovine embryonic stem cells, comprising the steps of:
a) building expression vectors comprising:
a1) at least one nucleotide sequence capable of expressing the human blood clotting factor IX gene and/or its fragments containing bovine signal peptide at the 5′ end; and
a2) at least a suitable promoter,
b) contacting bovine embryonic stem cells with the expression vectors from a).

2. A process for the purification of proteins generated by modified bovine embryonic stem cells comprising the steps of:
a) engaging the histidine tail to the 3′ end of at least one nucleotide sequence capable of expressing the human blood clotting factor IX gene and/or its fragments containing bovine signal peptide at the 5′ end; and
b) purifying the at least one nucleotide of a) thereby promoting the separation of biochemical compounds.
